(19) United States
(12) Patent Application Publication
(10) Pub. No.: US 2024/0002520 A1
(43) Pub. Date: Jan. 4, 2024

(54) TREATMENT OF CASTLEMAN DISEASE

(71) Applicant: THE TRUSTEES OF THE UNIVERSITY OF PENNSYLVANIA, Philadelphia, PA (US)

(72) Inventor: David C. FAJGENBAUM, Philadelphia, PA (US)

(73) Assignee: THE TRUSTEES OF THE UNIVERSITY OF PENNSYLVANIA, Philadelphia, PA (US)

(21) Appl. No.: 18/253,017

(22) PCT Filed: Nov. 15, 2021
(86) PCT No.: PCT/US21/72407

§ 371 Date: May 15, 2023

Related U.S. Application Data
(60) Provisional application No. 63/113,405, filed on Nov. 13, 2020.

(51) Int. Cl. C07K 16/28; G01N 33/68; A61K 31/436; A61K 39/395

(52) U.S. Cl. CPC C07K 16/2866; G01N 33/6893; A61K 31/436; A61K 39/3955; G01N 2333/521; G01N 2800/22; G01N 2800/52

(57) ABSTRACT

Provided are methods for assigning a subject having idiopathic multicentric Castleman disease (iMCD) to a group having a higher or lower probability of responding to treatment for iMCD using measured quantities of CXCL13. Also disclosed are methods of treating idiopathic multicentric Castleman disease (iMCD) in a subject in need thereof comprising administering to the subject an inhibitor of CXCL13 or of CXCR5. Also provided herein are methods for assigning a subject having idiopathic multicentric Castleman disease (iMCD) to a group having a higher or lower probability of responding to treatment for iMCD using measured quantities of specified biomarkers. The present disclosure also provides methods of treating idiopathic multicentric Castleman disease (iMCD) in a subject in need thereof comprising administering to the subject an inhibitor of the JAK-STAT3 pathway. Also disclosed are methods for assessing the absence or presence of iMCD in a subject.

### CROSS-REFERENCE TO RELATED APPLICATIONS

The present application claims the benefit of priority to U.S. Provisional Application No. 63/113,405, filed Nov. 13, 2020, the entire contents of which are incorporated herein by reference.

### TECHNICAL FIELD

The present disclosure pertains to methods for diagnosing and treating Castleman disease, and assays and methods for assessing the suitability of an ongoing or proposed treatment for a subject with Castleman disease.

### BACKGROUND

Idiopathic multicentric Castleman disease (iMCD) is a rare hematologic disorder with an estimated annual incidence of approximately 1,500 individuals in the United States and a 35-45% five-year overall mortality. 1-3 iMCD is one of three subtypes of multicentric Castleman disease (MCD), which also includes forms of MCD caused by uncontrolled human herpes virus-8 (HHV8) infection (HHV8-associated MCD) or associated with POEMS syndrome.⁴ Patients with iMCD present with a wide range of non-specific clinical and pathological features including cytokine-induced polyclonal lymphoproliferation, systemic inflammation, cytopenias, and multi-organ failure. No specific causes of iMCD have as yet been elucidated and the heterogeneous clinical presentation raises the possibility that multiple etiologies may exist. Many features of iMCD are observed in autoimmune, neoplastic, and infectious diseases, such as rheumatoid arthritis (RA), Hodgkin lymphoma (HL), and HHV8-associated MCD. 5 Specifically, auto-antibodies, a hallmark of autoimmune diseases, can be present in iMCD, Further, the lymphoproliferative pattern described in iMCD can mimic lymphoma, and intense episodes of acute inflammation, similar to a viral infection, often occur as well.

Though the etiology is unknown, interleukin-6 (IL-6) has been identified as a disease driver in a portion of patients.^{6,7} IL-6 is a pleiotropic cytokine that leads to activation of signaling pathways associated with survival and proliferation, most notably the Janus kinase/signal transducer and activator of transcription 3 (JAK-STAT3) pathway.⁸ Monoclonal antibodies directed against IL-6 (siltuximab) and the IL-6 receptor (tocilizumab) abrogate IL-6/IL-6Rα-induced signaling in iMCD.^{9,10} At present, siltuximab is the only FDA-approved therapy and recommended first-line.¹¹ However, 66% of iMCD patients treated in the siltuximab phase II registrational study did not meet primary response criteria, and pre-treatment IL-6 levels were not a strong predictor of response.^{10,12} Off-label monoclonal antibodies, such as rituximab, and cytotoxic chemotherapies are often tried for siltuximab non-responders, but these can have substantial toxicities as well as unclear efficacy.¹³ Overall, there are limited data to identify patients likely to respond to IL-6 blockade or discover novel therapeutic approaches.

CXCL13, a key regulator of lymph node germinal center development, was recently found to be the most elevated cytokine in iMCD flare, but the clinical significance of this finding is not yet clear. Interleukin-6 is the known driver of pathogenesis in a portion of patients and the target of the only FDA-approved treatment, siltuximab. In the Phase II study of siltuximab (NCT01024036), one-third of patients met response criteria, which were assessed after a minimum of 48 weeks. Early indicators of response to siltuximab are urgently needed to inform clinicians about the likelihood of patient response to therapy, adjust treatments if needed, and identify novel therapeutic targets for siltuximab non-responders.

### SUMMARY

Provided herein are methods for assigning a subject having idiopathic multicentric Castleman disease (iMCD) to a group having a higher or lower probability of responding to treatment for iMCD comprising comparing the amount of CXCL13 in a biological fluid obtained from the subject following commencement of the treatment to the amount of CXCL13 in a biological fluid obtained from the subject prior to commencement of the treatment; and, assigning the subject to a group having a higher probability of responding to the treatment if the amount of CXCL13 in the biological fluid obtained from the subject following commencement of the treatment represents a significant downward deviation relative to the amount of CXCL13 in the biological fluid obtained from the subject prior to commencement of the treatment.

Also disclosed herein are methods of treating idiopathic multicentric Castleman disease (iMCD) in a subject in need thereof comprising administering to the subject an inhibitor of CXCL13.

The present disclosure also provides methods of treating idiopathic multicentric Castleman disease (iMCD) in a subject in need thereof comprising administering to the subject an inhibitor of CXCR5.

Also provided herein are methods for assigning a subject having idiopathic multicentric Castleman disease (iMCD) to a group having a higher or lower probability of responding to treatment for iMCD comprising comparing the amount of biomarkers comprising one or more of APO E, SAP, iC3b, AREG, IgE, IL-6, and Epo in a biological fluid obtained from the subject prior to commencement of the treatment to reference values of the one or more biomarkers; and, assigning the subject to a group having a higher probability of responding to the treatment if the respective amounts of the one or more biomarkers in the biological fluid obtained from the subject prior to commencement of the treatment represent a significant deviation relative to the reference values for the one or more biomarkers.

The present disclosure also provides methods for assigning a subject having idiopathic multicentric Castleman disease (iMCD) to a group having a higher or lower probability of responding to treatment for iMCD comprising measuring the amount of biomarkers comprising one or more of APO E, SAP, iC3b, AREG, IgE, IL-6, and Epo in a biological fluid obtained from the subject prior to commencement of the treatment; and, assigning the subject to a group having a higher or lower probability of responding to treatment for iMCD using an optimized output of a function of the measured biomarkers in the biological fluid.

Also provided are method of treating idiopathic multicentric Castleman disease (iMCD) in a subject in need thereof comprising administering to the subject an inhibitor of the JAK-STAT3 pathway.

The present disclosure also provides methods for assessing the absence or presence of iMCD in a subject (i.e., assessing whether a subject has iMCD) comprising measuring the amount of CXCL13 in a biological fluid obtained from the subject, comparing the measured amount of CXCL13 in the biological fluid to a reference value corresponding to an amount of CXCL13 signifying an absence of iMCD, and assigning to the subject a positive diagnosis of iMCD if the measured amount of CXCL13 represents a significant upward deviation relative to the reference value of CXCL13.

### BRIEF DESCRIPTION OF THE DRAWINGS

FIG. 1 depicts an advocacy-industry-academic collaboration utilizing multiple technologies and platforms to perform precision medicine science on a collection of iMCD samples. Red boxes indicate sample collection, and blue boxes indicate scientific results.

FIGS. 2A and 2B provide a clustering analysis of serum proteomes of iMCD and related diseases, reveals a subgroup with a superior response to siltuximab. FIG. 2A provides a t-SNE plot visualizing serum proteomes of iMCD, Hodgkin lymphoma (lymphoma), HHV8-associated MCD (HHV8+MCD) and rheumatoid arthritis (RA) patients during active disease. Among the iMCD patients, siltuximab responders (partial response or complete response, per durable clinical and tumor/lymph node response criteria as determined in NCT01024036) are indicated with closed triangles, non-responders with open triangles, and patients for which siltuximab was not given as a monotherapy or response was not assessed by independent clinical trial review are represented by open circles. Colored lines are drawn around clusters as determined by elastic net with 5-fold cross validation. FIG. 2B depicts the top 40 serum analytes that best distinguish between clusters A-E, as selected by elastic net with 5-fold cross validation, across iMCD and related disease samples.

FIGS. 3A and 3B provide a clustering analysis of iMCD serum proteomes demonstrating 6 distinct clusters. FIG. 3A illustrates subtyping of iMCD patients into six clusters by elastic net clustering of iMCD samples using serum analyte levels, as measured by SOMAscan. Siltuximab responders (partial response or complete response, per durable clinical and tumor/lymph node response criteria as determined in NCT01024036) are indicated with closed triangles, non-responders with open triangles, and patients for which siltuximab was not given as a monotherapy or response was not assessed by independent clinical trial review are represented by open circles. Lines are drawn around clusters as determined by elastic net with 5-fold cross validation. FIG. 3B shows the proportion of patients within each cluster that demonstrated a partial or complete response to anti-IL-6 therapy when administered during active disease (as determined in NCT01024036.)

FIGS. 4A-4D illustrate a validation of a novel, proteomically definable iMCD subgroup that has superior response to siltuximab, increased disease activity, and elevated IL-6 levels. FIG. 4A provides a heat map of the 7 serum analytes that best distinguish Cluster-1 versus other clusters, as selected by elastic net with 5-fold cross validation, in the discovery dataset. FIG. 4B provides a correlation analysis between Cluster-1 score and response, disease activity, and IL-6 levels in the discovery cohort (two-sided p values). FIG. 4C is a heat map of the 7 serum analytes tested in an independent validation dataset. FIG. 4D provides a correlation analysis between Cluster-1 score and response, disease activity, and IL-6 levels in the validation cohort (one-sided p values). Box plots show center median, first and third quartile, and whiskers extend to 1.5*interquartile range. Cluster-1 scores are scaled from 0 to 1 for each cohort.

FIGS. 5A-5D show the results of immunohistochemistry of pSTAT3 in iMCD and normal control lymph nodes. FIG. 5A provides how iMCD demonstrated significantly more positive staining in the interfollicular space compared with normal lymph nodes (p=0.0037). No significant differences were observed in germinal centers (p=0.2610), secondary follicles (p=0.4119), and mantle zones (p=0.552). As provided in FIG. 5B, within the interfollicular space, iMCD lymph nodes demonstrated significantly higher weak (p=0.014) and medium (p=0.0066) with no difference in strong staining intensity. Representative images of a (C) normal lymph node (FIG. 5C) and an iMCD lymph node (FIG. 5D) at 40× magnification are provided.

FIG. 6 provides coefficient estimates of the changes in analytes between patients who responded to anti-IL-6 antibody (siltuximab) compared to those who did not respond to siltuximab at day 8 plotted against those at day 64 for 1178 analytes measured in patients at day 0, day 8, and day 64 following siltuximab administration.

FIG. 7 depicts CXCL13 serum protein levels over time during treatment with anti-IL-6 antibody among patients who responded to anti-IL-6 antibody (siltuximab; responders) compared to those who did not respond to siltuximab (nonresponders) and patients treated with placebo.

FIG. 8 illustrates the log 2(fold change) in CXCL13 levels compared to baseline at day 8 and day 64 (Cycle 4/Day 1).

FIG. 9 provides a volcano plot showing BLC/CXCL13 as the third most up-regulated serum protein and the top cytokine in iMCD versus healthy controls.

FIG. 10 is a bar graph showing BLC/CXCL13 as the most up-regulated serum protein when comparing the 97.5th percentile of iMCD patients versus the top 97.5th percentile of healthy controls.

FIG. 11 provides a heatmap of proteins, including CXCL13/BLC, demonstrating significant differences in expression between iMCD, RA, HL, and HHV8-M.

FIG. 12A illustrates a plot of CXCL13 levels between iMCD and multiple myeloma, and FIG. 12B provides the reporter operator curve demonstrating the sensitivity and specificity of CXCL13 levels to distinguish iMCD from multiple myeloma.

FIG. 13 provides the discovery cohort used to identify proteins with significant changes between pre-treatment and post-treatment. Logistic regression was used to determine the effect of CXCL13 percent reduction by day 8 on response status. Response˜percent reduction in CXCL13. The model was compared with a model including age, sex, and baseline CRP as covariates. The covariate model did not outperform the simple model, so the simple model was selected for interpretability.

FIG. 14 shows the discovery cohort used to demonstrate that reduction in CXCL13 has a significant effect on response status and is thus as an early indicator of response to siltuximab. NR=non-response; R=response.

FIG. 15 depicts the reporter operator curve used to identify an ideal threshold for identifying likely responders versus non-responders based on reduction in serum levels of CXCL13 in the discovery cohort (AUC=0.86). The point makes the decision rule to be a perfect classifier, and the optimal point with threshold p=0.379, which corresponds to a 17% reduction in CXCL13.

FIG. 16 illustrates the classification by logistic regression in the discovery cohort and the performance of the optimal threshold to predict response. Responders plotted along the top horizontal bar and non-responders along the bottom horizontal bar. The x-axis represents the percent change in CXCL13 from baseline by day 8. The logistic regression curve is plotted in blue, with a horizontal line drawn at the probability of best classification, which intersects the curve at 17% reduction. A value of p>0.379 is predicted to respond, and a value of less than 0.379 is predicted not to respond. For threshold p=0.379, this corresponds to a 17% reduction in CXCL13 levels between day 0 and day 8. Therefore, a >17% reduction in CXCL13 has a 79% accuracy, 82% recall, and 67% precision in response prediction in the discovery cohort. False positive rate=23%; true positive rate=82%.

FIG. 17A illustrates the change over time in CXCL13 levels from pre-treatment to Day 22/29 and Day 43 between siltuximab responders and non-responders in an independent cohort from the phase I siltuximab trial. FIG. 17B provides the mean (95% CI) expression levels of CXCL13 over time.

FIG. 18A depicts the reporter operator curve demonstrating the performance of reduction in serum levels of CXCL13 for identifying likely responders versus non-responders in the validation cohort (AUC=1.00), and FIG. 18B provides a bar plot of response prediction values colored by true response status illustrates the perfect separation between responders and non-responders in the validation set when using this biomarker.

FIG. 19 illustrates the classification by logistic regression in the validation cohort and the performance of the threshold to predict response. Responders plotted along the top horizontal bar and non-responders along the bottom horizontal bar. The x-axis represents the percent change in CXCL13 from baseline by day 22/29. The logistic regression curve is plotted in blue, with a horizontal line drawn at the threshold of best classification from the discovery cohort (17% reduction). A >17% reduction in CXCL13 levels has a 90% accuracy, 100% recall, and 82% precision in response prediction in the validation cohort. False positive rate=18%; true positive rate=100%.

FIG. 20 depicts how geneset enrichment analysis was performed on RNA sequencing data from circulating immune cell samples from iMCD patients treated with serum only versus serum plus rhCXC13 as well as iMCD patients treated with serum only versus serum plus rhCXCL13 and anti-CXCL13. Pathways previously found to be up-regulated in iMCD patients were found to be up-regulated in a patient sample treated with rhCXCL13 and also down-regulated in the patient's sample treated with rhCXCL13 and anti-CXCL13.

FIG. 21 further illustrates how pathways previously found to be up-regulated in iMCD patients were found to be up-regulated in a patient sample treated with rhCXCL13 and also down-regulated in the patient sample treated with rhCXCL13 and anti-CXCL13.

### DETAILED DESCRIPTION OF ILLUSTRATIVE EMBODIMENTS

The present invention may be understood more readily by reference to the following detailed description taken in connection with the accompanying examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

The disclosures of each patent, patent application, and publication cited or described in this document are hereby incorporated herein by reference, in their entirety.

As employed above and throughout the disclosure, the following terms and abbreviations, unless otherwise indicated, shall be understood to have the following meanings.

In the present disclosure the singular forms “a”, “an”, and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. Furthermore, when indicating that a certain chemical moiety “may be” X, Y, or Z, it is not intended by such usage to exclude other choices for the moiety; for example, a statement to the effect that R1 “may be alkyl, aryl, or amino” does not exclude other choices for R1, such as halo, aralkyl, and the like.

When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” may refer to a value of 7.2 to 8.8, inclusive; as another example, the phrase “about 8%” may refer to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.”

A “biological fluid” may be whole blood, serum, plasma, or any other fluid derived from the subject in question.

Throughout the present disclosure, where the term “idiopathic multicentric Castleman disease” or “iMCD” is used, such not necessarily intended to be limited to any particular form of Castleman disease. Accordingly, the terms “idiopathic multicentric Castleman disease” and “iMCD” can be read as embracing any form of Castleman disease, including, for example, unicentric Castleman disease and HHV8+MCD.

As described herein, using serum proteomic analysis using a multiplex DNA-aptamer-based platform, the present inventors have identified a novel iMCD subgroup with superior response to treatment, such as administration of anti-IL-6 therapy (e.g., siltuximab), that was validated using an independent cohort and orthogonal platform. The inventors further leveraged the proteomic data to identify novel candidate pathways involved in iMCD pathogenesis, some of which are known targets for FDA-approved drugs. In addition, JAK-STAT3 was validated as a therapeutic target using orthogonal methods.

Additionally, it was found that CXCL13, along with several other proteins that demonstrated significant decline following treatment for iMCD, including IgA and beta2-microglobulin, can be routinely measured and could serve as indicators of the likelihood of response soon after commencing therapy. These proteins also represent a more continuous scale of response than traditional outcome measures. Given that iMCD may have a sudden and severe onset, the presently disclosed early indicators of response to iMCD therapy, including anti-IL6 therapy, are critical for timely treatment administration.

Accordingly, disclosed herein are methods for assigning a subject having idiopathic multicentric Castleman disease (iMCD) to a group having a higher or lower probability of responding to treatment for iMCD comprising comparing the amount of CXCL13 in a biological fluid obtained from the subject following commencement of the treatment to the amount of CXCL13 in a biological fluid obtained from the subject prior to commencement of the treatment; and, assigning the subject to a group having a higher probability of responding to the treatment if the amount of CXCL13 in the biological fluid obtained from the subject following commencement of the treatment represents a significant downward deviation relative to the amount of CXCL13 in the biological fluid obtained from the subject prior to commencement of the treatment.

The biological fluid obtained from the subject following commencement of the treatment may be obtained from about two days to about three months following commencement of the treatment. For example, the biological fluid may be obtained about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, or 60 days following commencement of the treatment. In certain embodiments, the biological fluid obtained at least or about one week from the subject following commencement of the treatment. More than one sample of biological fluid may be obtained, and the respective samples may be at different times following commencement of the treatment, and the comparison to the biological fluid obtained from the subject prior to commencement of the treatment may be made with respect to each of the samples that are obtained following commencement of treatment.

The treatment that to which the subject is subjected may be any single or combination of treatments for iMCD. The treatment may be, for example, anti-IL-6 therapy, such as administration of siltuximab to the subject. Any treatment for iMCD that is disclosed herein may be the treatment to which the subject is subjected pursuant to the present methods.

In accordance with any embodiment disclosed herein, a significant downward deviation of the amount of CXCL13 may be equivalent to a downward deviation of greater than about 10%, such as about 10-30% or greater, of CXCL13. In some embodiments, the significant downward deviation is equivalent to a downward deviation of about 15-20% of CXCL13, or greater. For example, the significant downward deviation may be equivalent to a downward deviation of about 10, 11, 12, 13, 14, 15, 16, 17, 18, 19, 20, 21, 22, 23, 24, 25, 26, 27, 28, 29, 30% of CXCL13, or greater.

In accordance with the present methods, when the subject is assigned to a group having a lower probability of responding to the treatment, the method may comprise reducing or ceasing the treatment or adding a new treatment following the assignment. Likewise, when the subject is assigned to a group having a higher probability of responding to the treatment, the method may further comprise continuing the treatment following the assignment.

Also disclosed herein are methods of treating idiopathic multicentric Castleman disease (iMCD) in a subject in need thereof comprising administering to the subject an inhibitor of CXCL13. The inhibitor of CXCL13 may be, for example, VX5 antibody (Vaccinex, Rochester, NY), JT03 (Jyant Technologies, Marietta, GA), or TJX7 (I-Mab Biopharma Co., Gaithersburg, MD). The present methods may further comprise administering to the subject a further treatment for iMCD. For example, the methods may include administering to the subject a further treatment for iMCD at least partially during administration of the inhibitor of CXCL13 to the subject, at least partially prior to administration of the inhibitor of CXCL13 to the subject, at least partially following administration of the inhibitor of CXCL13 to the subject, or any combination thereof. The further treatment for iMCD may be, for example, an inhibitor of IL-6, an inhibitor of CXCR5, an inhibitor of JAK protein, an inhibitor of the JAK/STAT3 pathway, or any combination thereof.

Also disclosed herein are methods of treating idiopathic multicentric Castleman disease (iMCD) in a subject in need thereof comprising administering to the subject an inhibitor of CXCR5. The inhibitor of CXCR5 may be, for example, SAR113244 antibody. The present methods may further comprise administering to the subject a further treatment for iMCD. For example, the methods may include administering to the subject a further treatment for iMCD at least partially during administration of the inhibitor of CXCR5 to the subject, at least partially prior to administration of the inhibitor of CXCR5 to the subject, at least partially following administration of the inhibitor of CXCR5 to the subject, or any combination thereof. The further treatment for iMCD may be, for example, an inhibitor of IL-6, an inhibitor of CXCL13, an inhibitor of JAK protein, an inhibitor of the JAK/STAT3 pathway, or any combination thereof.

Also provided herein are methods for assigning a subject having idiopathic multicentric Castleman disease (iMCD) to a group having a higher or lower probability of responding to treatment for iMCD comprising comparing the amount of biomarkers comprising one or more of APO E, SAP, iC3b, AREG, IgE, IL-6, and Epo in a biological fluid obtained from the subject during or prior to commencement of the treatment to reference values of the one or more biomarkers; and, assigning the subject to a group having a higher probability of responding to the treatment if the respective amounts of the one or more biomarkers in the biological fluid obtained from the subject during or prior to commencement of the treatment represent a significant deviation relative to the reference values for the one or more biomarkers. Depending on the specific biomarker at issue, deviation may be upward or downward relative to the reference value.

For embodiments in which the biological fluid is obtained from the subject during some point following commencement of the treatment, the biological fluid may be obtained from about two days to about three months following commencement of the treatment. For example, the biological fluid may be obtained about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, or 60 days following commencement of the treatment. In certain embodiments, the biological fluid is obtained at least or about one week from the subject following commencement of the treatment. More than one sample of biological fluid may be obtained, and the respective samples may be at different times following commencement of the treatment, and the comparison to the biological fluid obtained from the subject prior to commencement of the treatment may be made with respect to each of the samples that are obtained following commencement of treatment.

In some embodiments, the comparison is made between a single one of the biomarkers in a biological fluid obtained from the subject during or prior to commencement of the treatment to a reference value for that biomarker. In other embodiments, the comparison is made between two, three, four, five, six, or all seven of the biomarkers in a biological fluid obtained from the subject during or prior to commencement of the treatment to a corresponding reference values for the respective biomarkers.

The reference value for a given biomarker may be derived from a general population of subjects with iMCD, from a population of subjects in which iMCD is known to be absent, or some other measured, calculated, or projected value representing a baseline from which an upward deviation is indicative of a higher probability of responding to the treatment for iMCD.

In some embodiments in which the comparison is made between all seven of the biomarkers in a biological fluid obtained from the subject during or prior to commencement of the treatment and corresponding reference values for the respective biomarkers, the respective biomarkers may be assigned weighting coefficients. For example, the respective biomarkers may be assigned weighting coefficients according to the following algorithm:

| (Intercept) | Apo E | SAP | Ic3b | AREG | IgE | IL-6 | Epo |
|---|---|---|---|---|---|---|---|
| −0.42321247 | −0.19178761 | 0.27776701 | 0.11668378 | −0.13851479 | 0.04776587 | 0.01795248 | 0.03293765 |

The present disclosure also provides methods for assigning a subject having idiopathic multicentric Castleman disease (iMCD) to a group having a higher or lower probability of responding to treatment for iMCD comprising measuring the amount of biomarkers comprising one or more of APO E, SAP, iC3b, AREG, IgE, IL-6, and Epo in a biological fluid obtained from the subject during or prior to commencement of the treatment; and, assigning the subject to a group having a higher or lower probability of responding to treatment for iMCD using an optimized output of a function of the measured biomarkers in the biological fluid.

For embodiments in which the biological fluid is obtained from the subject during some point following commencement of the treatment, the biological may be obtained from about two days to about three months following commencement of the treatment. For example, the biological fluid may be obtained about 2, 3.4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, or 60 days following commencement of the treatment. In certain embodiments, the biological fluid obtained at least or about one week from the subject following commencement of the treatment. More than one sample of biological fluid may be obtained, and the respective samples may be at different times following commencement of the treatment, and the comparison to the biological fluid obtained from the subject prior to commencement of the treatment may be made with respect to each of the samples that are obtained following commencement of treatment.

In some embodiments, the comparison is made between a single one of the biomarkers in a biological fluid obtained from the subject during or prior to commencement of the treatment to a reference value for that biomarker. In other embodiments, the comparison is made between two, three, four, five, six, or all seven of the biomarkers in a biological fluid obtained from the subject during or prior to commencement of the treatment to a corresponding reference values for the respective biomarkers.

The reference value for a given biomarker may be derived from a general population of subjects with iMCD, from a population of subjects in which iMCD is known to be absent, or some other measured, calculated, or projected value representing a baseline from which an upward deviation is indicative of a higher probability of responding to the treatment for iMCD.

In some embodiments in which the comparison is made between all seven of the biomarkers in a biological fluid obtained from the subject during or prior to commencement of the treatment and corresponding reference values for the respective biomarkers, the respective biomarkers may be assigned weighting coefficients. For example, the biomarkers may be assigned weighting coefficients according to the following algorithm:

| (Intercept) | Apo E | SAP | Ic3b | AREG | IgE | IL-6 | Epo |
|---|---|---|---|---|---|---|---|
| −0.42321247 | −0.19178761 | 0.27776701 | 0.11668378 | −0.13851479 | 0.04776587 | 0.01795248 | 0.03293765 |

Also provided are methods of treating idiopathic multicentric Castleman disease (iMCD) in a subject in need thereof comprising administering to the subject an inhibitor of the JAK-STAT3 pathway. In some embodiments, the method comprises administering to the subject an inhibitor of the JAK protein. The methods may further comprise administering to the subject a further treatment for iMCD. For example, the methods may include administering to the subject a further treatment for iMCD at least partially during administration of the inhibitor of the JAK-STAT3 pathway to the subject, at least partially prior to administration of the inhibitor of the JAK-STAT3 pathway to the subject, at least partially following administration of inhibitor of the JAK-STAT3 pathway to the subject, or any combination thereof. The further treatment for iMCD may be, for example, an inhibitor of IL-6, an inhibitor of CXCR5, or an inhibitor of CXCL13.

The present disclosure also provides methods for assessing the absence or presence of iMCD in a subject (i.e., assessing whether a subject has iMCD) comprising measuring the amount of CXCL13 in a biological fluid obtained from the subject, comparing the measured amount of CXCL13 in the biological fluid to a reference value corresponding to an amount of CXCL13 signifying a lower or higher likelihood of iMCD, and assigning to the subject a higher likelihood of a positive diagnosis of iMCD if the measured amount of CXCL13 represents a significant upward deviation relative to the reference value of CXCL13 and a lower likelihood of a positive diagnosis of iMCD if the measured amount of CXCL13 does not represent a significant upward deviation relative to the reference value of CXCL13.

Pursuant to such methods the reference value for CXCL13 may be derived from a subject or a population of subjects in whom iMCD is known to be absent, or some other measured, calculated, or projected value representing a baseline from which an upward deviation is indicative of a higher probability of the presence of iMCD.

In other embodiments, the reference value for CXCL13 could be derived from a subject or a population of subjects in whom iMCD is known to be present, thereby representing a threshold from which an upward deviation is indicative of a higher probability of the presence of iMCD, and from which a downward deviation could be indicative of a lower probability of the presence of iMCD.

A significant upward deviation of the amount of CXCL13 may be equivalent to an upward deviation of about 25% or greater, such as about 25, 30, 35, 40, 45, 50, 55, 60, 65, 70, 75, 80, 85, 90, 95, 100% or greater, of CXCL13, relative to the reference value. In some embodiments, the significant upward deviation is equivalent to an upward deviation of about 200%, or greater, relative to the reference value. For example, the significant upward deviation may be equivalent to an upward deviation of about 2, 3, 4, 5, 6, 7, 8, 9, 10, 11, 12, 13, 14, 15, 16, 17, 18, 19, or 20 times the reference value of CXCL13, or greater.

In accordance with the disclosed methods for assessing the absence or presence of iMCD in a subject, if the measured amount of CXCL13 from the biological fluid of the subject represents a significant upward deviation relative to the reference value of CXCL13, the method may further comprise treating the subject for iMCD. The treatment for iMCD may include any of the presently disclosed, previous known therapies, such as administering to the subject an inhibitor of CXCL13, an inhibitor of CXCR5, an inhibitor of IL-6, an inhibitor of JAK protein, an inhibitor of the JAK/STAT3 pathway, or any combination thereof. The subject may also or alternatively be treated using a modality that is later developed and not yet known at the time of the present disclosure.

### EXAMPLES

The present invention is further defined in the following Examples. It should be understood that these examples, while indicating preferred embodiments of the invention, are given by way of illustration only, and should not be construed as limiting the appended claims. From the above discussion and these examples, one skilled in the art can ascertain the essential characteristics of this invention, and without departing from the spirit and scope thereof, can make various changes and modifications of the invention to adapt it to various usages and conditions.

### Example 1—Discovery and Validation of a Novel Subgroup and Therapeutic Target in Idiopathic Multicentric Castleman Disease

Proteomic quantification of 1,178 analytes was performed on serum of 88 iMCD patients, 60 patients with clinico-pathologically overlapping diseases (human herpesvirus-8(HHV8)-associated MCD, N=20; Hodgkin lymphoma, N=20; rheumatoid arthritis, N=20), and 44 healthy controls. Unsupervised clustering revealed iMCD patients have heterogeneous serum proteomes that did not cluster with clinico-pathologically overlapping diseases. Clustering of iMCD patients identified a novel subgroup with superior response to siltuximab, which was validated using a 7-analyte panel (apolipoprotein E, amphiregulin, serum amyloid P-component, inactivated complement C3b, immunoglobulin E, IL-6, erythropoietin) in an independent cohort. Enrichment analyses and immunohistochemistry identified-JAK-STAT3 signaling as a therapeutic target in both siltuximab responders and non-responders. These results indicate that targeting JAK-STAT3 signaling with a JAK inhibitor represent a viable treatment approach for siltuximab non-responders.

### Materials and Methods

### Proteomics Samples and Clinical Data

For the discovery cohort, samples were obtained from 88 iMCD patients, with N=73 pre-treatment disease flare samples collected as part of the siltuximab phase II study (NCT01024036) and N=15 disease flare samples collected in real-world practice from 6 sites. Samples collected in real-world practice were included to better represent the full spectrum of iMCD. We obtained samples from 60 patients with diseases that overlap in clinical and pathological presentation with iMCD, including HHV8-associated MCD (N=20), HL (N=20), and RA (N=20), which are caused by viral, neoplastic, and autoimmune mechanisms, respectively, and 44 healthy individuals. An independent cohort of 23 iMCD patients enrolled in the siltuximab phase I study (NCT00412321) served as a validation cohort. All serum samples from the phase I and the phase II studies were collected and processed following respective standardized protocols.

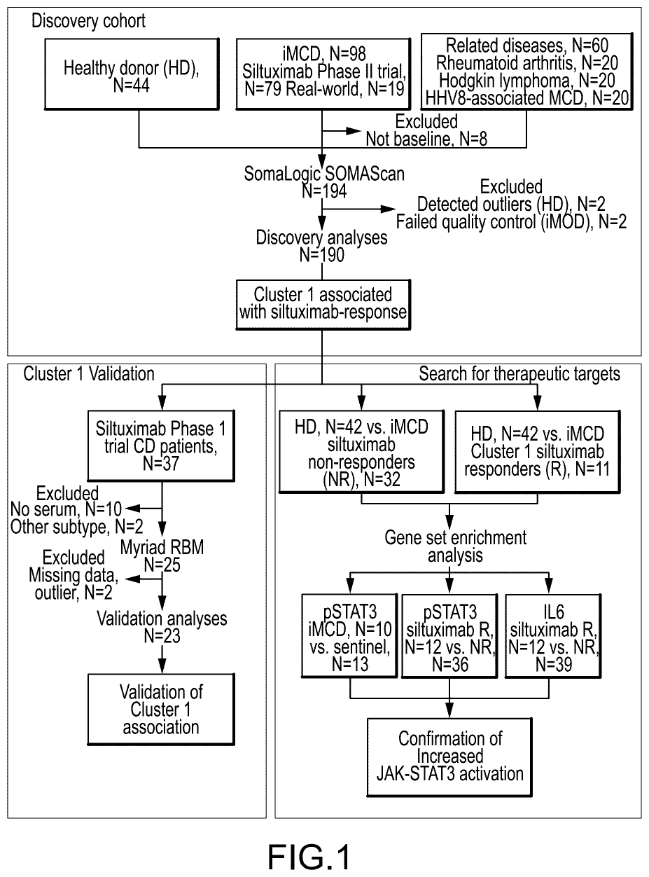

Clinical and laboratory data were collected at the time of sample draw for iMCD patients. To assess disease activity, we adapted a previously published disease activity score using C-reactive protein, hemoglobin, and albumin. 14 Response to siltuximab was assessed by durable symptomatic and tumor response criteria (no worsening in 34 MCD overall symptom scale and at least a partial response by Cheson criteria) 15 for patients in the phase II study and by tumor response criteria (at least a partial response by Cheson criteria) 15 for patients in the phase I study. All patients provided informed consent, and the research was approved by the Quorum Review Institutional Review Board. Study flow and clinical characteristics of iMCD patients in the discovery and validation cohorts are shown in FIG. 1.

### Proteomics Platforms

For the samples in the discovery cohort, SomaLogic SOMAscan was used to measure 1,305 serum analytes by DNA-based aptamer technology¹⁶, of which 1,178 passed quality control and were included in analyses of the discovery dataset.¹⁷ Each analyte was log 2 transformed and capped at the 2.5th and 97.5th percentiles.

For the 23 samples in the validation cohort, Rules Based Medicine (RBM) DiscoveryMap v1.0 was used to measure 190 serum analytes by a microsphere-based, multiplexed immunoassay platform.¹⁸ RBM values were converted to standardized units and log 2 transformed. Values below the per-target least detectable dose were truncated to the least detectable dose. Of 190 proteins measured by RBM, 154 can be mapped to targets in the Somalogic platform, and 140 remained for analysis after filtering out low-quality targets on both platforms.

### Gene Set Enrichment Analysis (GSEA)

To identify enriched pathways, GSEA, utilizing the Hallmark database, was performed between a subset of iMCD patients Cluster-1, who responded to siltuximab therapy, and healthy controls as well as between all iMCD siltuxmab non-responders and healthy controls. 19 Of the 1,178 proteins that passed quality control, N=1,139 mapped to a unique gene and were included. The threshold for significance for the false discovery rate was 0.20.

### Immunohistochemistry

To investigate pSTAT3 expression in iMCD compared to healthy controls, we collected formalin fixed paraffin embedded (FFPE) lymph node tissue from 10 iMCD patients enrolled in the ACCELERATE Natural History Registry (NCT02817997) and from 15 breast cancer patients with non-metastatic sentinel lymph nodes (normal control). HL patients (N=13) were selected as positive controls for assay validation.²⁰ IHC staining was performed on a Leica Bond Max automated staining system (Leica Biosystems) using the Bond intense R staining kit (Leica Biosystems DS9263). Following a standard protocol, pSTAT3(Tyr 705) antibody (Cell Signaling, 9145) was used to stain formalin fixed paraffin embedded tissue slides. Slides were digitally scanned at 20× magnification on an Aperio ScanScope CS-O slide scanner (Leica Biosystems).

Secondary follicles, germinal centers, mantle zones, and randomly selected sections of interfollicular space were annotated and audited by independent blinded researchers using Aperio ImageScope, and analysis was performed using Image Analysis Toolkit Software color deconvolution v9 algorithm. The percentage weak, medium, strong and no staining were collected for each region, and data were centered log-ratio transformed. Wilcoxon rank-sum tests were performed to compare staining intensity between iMCD and control and between HL and control. When appropriate, p-values were Bonferroni corrected.

To identify differential expression of IL-6 and pSTAT3 expression in siltuximab responders and non-responders, we examined IHC data from 51 and 48 iMCD patients in the phase II siltuximab study, respectively. FFPE tissue samples were obtained from patients in the phase II study prior to initiation of treatment and were processed according to a standard protocol (Supplementary Methods).

### Statistical Analysis

Data analysis was performed using the Medidata Rave Omics machine learning platform and R v3.4.4. To identify sample outliers within disease categories, principal component analysis reconstruction residual, average pairwise distance (APW), and the APW to the K-nearest neighbors were used. Data points identified by at least two methods were considered outliers and removed from analysis.

The t-SNE algorithm as implemented in the Rtsne package was used to visualize a 2D representation of the high dimensional protein expression data Elastic net classifiers were fit using the glmnet R package. The number of features (protein targets) selected was determined by performing 5-fold cross-validation and selecting the smallest number of features such that the overall cross-validation error was within 1 standard error of the minimum. An elastic net classifier was used to predict Cluster-1 membership in the discovery cohort using only those Somalogic SomaSCAN analytes that could be mapped to equivalent proteins in the RBM platform. The fit coefficients (apolipoprotein E (Apo E): −0.191788; serum amyloid P-component (SAP): 0.277767; inactivated complement C3b (iC3b): 0.116684; amphiregulin (AREG): −0.138515; immunoglobulin E (IgE): 0.047766; IL-6: 0.017952; erythropoietin (Epo): 0.032938) were used to calculate Cluster-1 score in both the discovery and validation cohorts. A one-sided test was used to test positive association between Cluster-1 score and response, disease activity, and IL-6 levels in the validation cohort, because the discovery studies led us to hypothesize that there would be a positive association. All other p values are two-sided with α=0.05.

### Results

iMCD is a Heterogeneous Disorder Compared to Related Inflammatory and Neoplastic Disorders

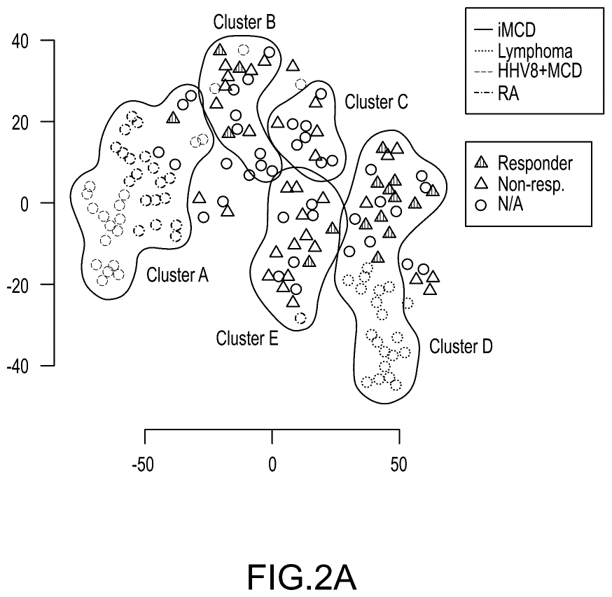

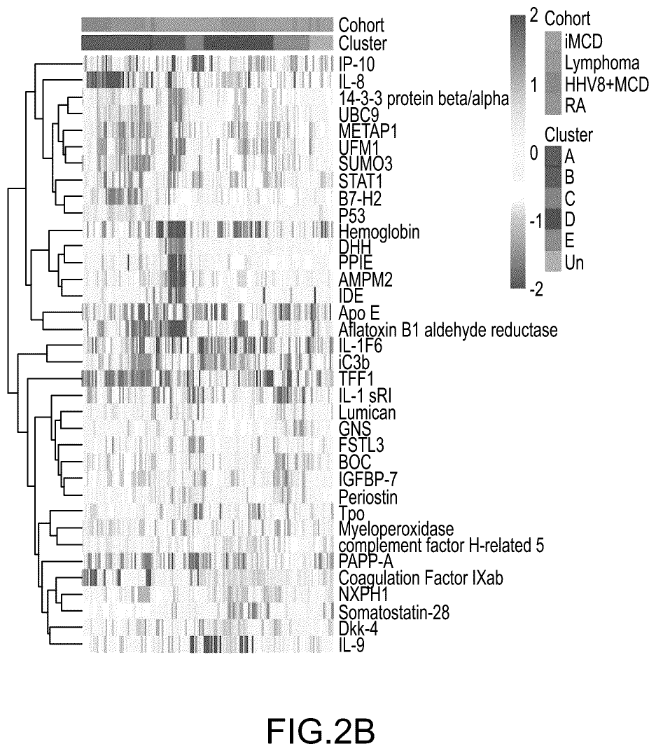

To characterize the serum proteome of iMCD in the context of HL, RA, and HHV8-associated MCD, we applied an unbiased elastic net and hierarchical clustering algorithm (FIG. 2A). We hypothesized that the iMCD samples (N=88) would cluster together or close to a single related disease, which could indicate overlapping etiological or pathophysiological mechanisms. Each of the comparator diseases formed a clear group, while most iMCD samples occupied the space between the comparator diseases. We identified five distinct clusters composed of 134 samples (FIG. 2B); 14 samples were unclustered. Interestingly, iMCD samples were present in all five clusters and in the unclustered group. More than half of the iMCD samples (49/88) were included in Clusters-B, -C, and -E, which together only contained 3 comparator disease samples. Cluster-D contained nearly all of the HL (19/20) samples and the greatest proportion (22/88, 25%) of iMCD samples in a single cluster. Cluster-A contained nearly all RA (19/20) and HHV8-associated MCD (17/20) samples as well as 5/88 iMCD samples. These results indicate that iMCD is highly heterogeneous with proteomic profiles similar to autoimmune, infectious and neoplastic diseases in some cases but not others.

Identification of a Novel iMCD Subgroup with a Superior Response to Siltuximab

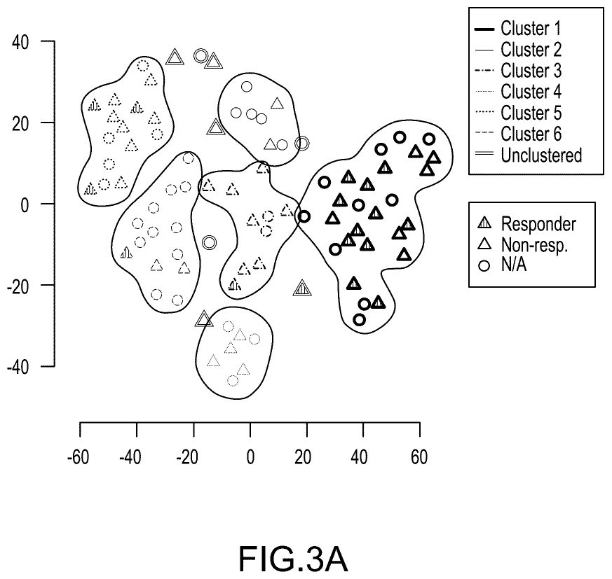

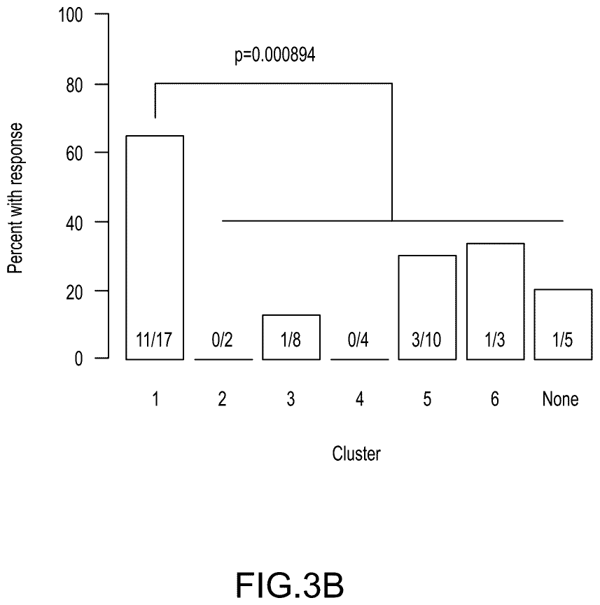

Due to the heterogeneity observed across iMCD samples when clustered with comparator diseases, we next performed unbiased clustering among only iMCD samples to discover clinically meaningful subgroups. The algorithm identified six proteomically-defined clusters that ranged in size from seven to 27 samples (FIG. 3A). No significant associations with race, sex, age, concurrent or prior corticosteroid use, prior use of antineoplastic or immunosuppressive drugs, or processing batch were observed. Siltuximab response was assessed for patients from the phase II study who had an independent response assessment performed.¹⁰ Compared to all other patients, patients represented in Cluster-1 demonstrated significantly higher disease activity (p=7.062×10⁻⁹), significantly higher baseline IL-6 levels as measured by SomaSCAN assay (p=5.709×10⁻⁹), and significantly higher response to siltuximab (65% (11/17) vs 19% (6/32); p=8.94×10⁻⁴) (FIG. 3B). Interestingly, the Cluster-1 iMCD patients represented all of the iMCD patient samples that clustered with HL patients in Cluster-D (FIG. 2A, FIG. 3A). These results demonstrate that there may be a proteomically-distinct iMCD subgroup that is identifiable prior to treatment and has a superior response to anti-IL-6 therapy.

Validation of a Novel Subgroup of iMCD with a Superior Response to Siltuximab

To validate the identification of this novel iMCD subgroup with a superior response to siltuximab in our discovery dataset, serum samples from an independent cohort of 23 iMCD patients enrolled in the phase I clinical trial of siltuximab were analyzed using an orthogonal targeted proteomic panel (RBM) of 190 analytes.²¹ Mean protein levels as measured on both platforms were strongly associated (p=3.35×10⁻¹³), suggesting cross-validity of results across the assays.

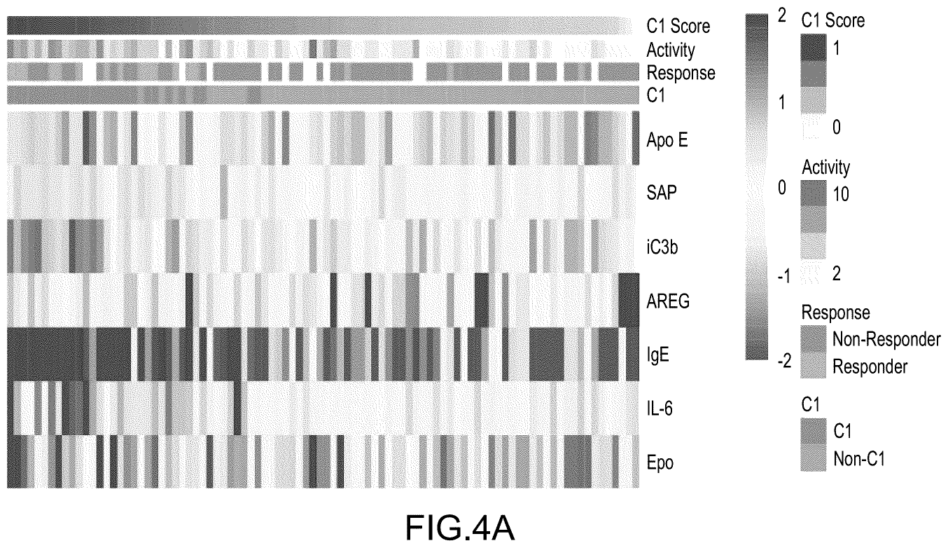

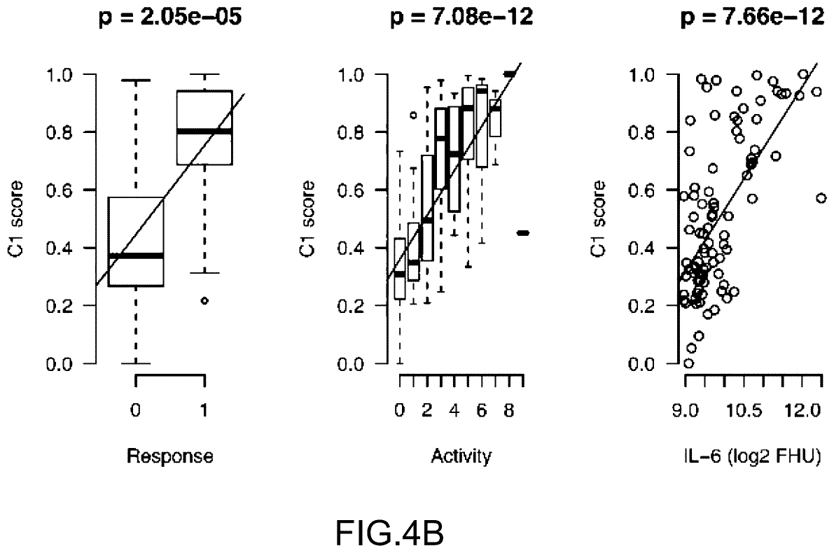

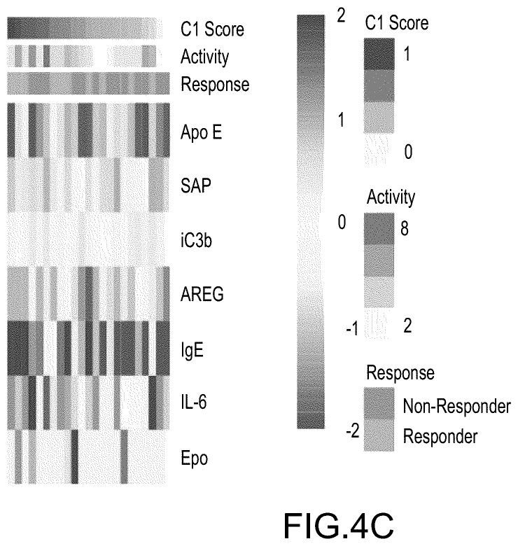

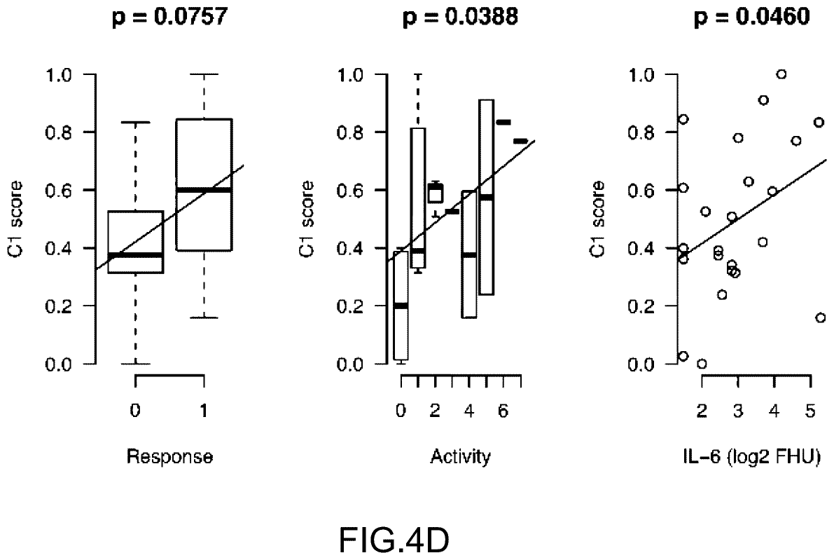

To determine whether Cluster-1 inclusion was predictive of siltuximab response in the validation cohort, we derived a “Cluster-1 score” using an elastic net to determine the fewest proteins present on both platforms and that most effectively predicted Cluster-1 membership in the discovery dataset. The derived Cluster-1 score includes ApoE, AREG), SAP, iC3b, IgE, IL-6, and Epo. Among samples in the discovery dataset, Cluster-1 score was significantly associated with siltuximab response (p=2.05×10⁻⁵), disease activity (p=7.08×10⁻¹²), and clinically-obtained IL-6 level (p=3.37×10⁻⁶) (FIG. 4A-B). We hypothesized that Cluster-1 score would likewise be positively associated with response, disease activity, and IL-6 levels when applied to the validation cohort. There was a trend towards a positive association between Cluster-1 score and siltuximab response (p=0.0757), and Cluster-1 score was significantly associated with increased disease activity (p=0.0388) and IL-6 levels (p=0.0460) (FIG. 4C-D). Despite notable differences in the proteomic technique and response criteria used (11, 14) in the discovery and validation cohorts, these results validate the discovery of an iMCD subtype with superior response to siltuximab

### Identification of JAK-STAT3 as a Candidate Driver Pathway in Siltuximab Non-Responders

Next, we sought to utilize the discovery proteomic dataset to identify candidate driver pathways and potential therapeutic targets for siltuximab non-responders. As a proof of principle, we performed GSEA on the proteomic data from Cluster-1 siltuximab responders compared to healthy controls. We hypothesized that IL-6-JAK-STAT3 signaling would be significantly enriched as IL-6 signaling is an essential disease driver in patients who improve with siltuximab. As expected, IL-6-JAK-STAT3 signaling was significantly enriched below our threshold (q=0.184) along with four other pathways (Table 1).

TABLE 1
Hallmark pathways significantly enriched in the
discovery dataset among Cluster-1 anti-IL6 responders
and in all siltuximab non-responders

| | | |
|---|---|---|
| Pathway | Nominal P value | FDR q-value |

Enriched pathways in Cluster-1 siltuximab responders vs HDs

| | | |
|---|---|---|
| TNFa signaling via NFkB | 0.004 | 0.090 |
| Estrogen Response Early | 0.013 | 0.137 |
| IFN gamma response | 0.033 | 0.149 |
| Allograft Rejection | 0.033 | 0.167 |
| Signature | | |
| IL-6-JAK STAT3 | 0.020 | 0.184 |
| Signaling | | |

Enriched pathways in siltuximab non-responders vs HDs

| | | |
|---|---|---|
| KRAS Signaling Up | 0.029 | 0.118 |
| IL-6-JAK STAT3 | 0.031 | 0.144 |
| Signaling | | |
| TNFa signaling via NFkB | 0.006 | 0.173 |
| Allograft Rejection | 0.043 | 0.177 |
| Signature | | |
| IL2 STAT5 Signaling | 0.018 | 0.179 |

Next, GSEA was repeated for non-responders in the discovery dataset. As seen in Cluster-1 responders, IL-6-JAK-STAT3 signaling (q=0.144), TNFα signaling via NFκB (q=0.173), and allograft rejection signature (q=0.177) were significantly enriched in non-responders. In addition, IL-2-STATS signaling (q=0.177) and KRAS signaling up (q=0.118) were identified as significantly enriched (Table 1). Several of the pathways identified in patients either with or without a response to siltuximab can be targeted with existing FDA approved compounds. 22-32) The results of the GSEA analysis therefore provide a rationale for further investigation of these approaches in iMCD.

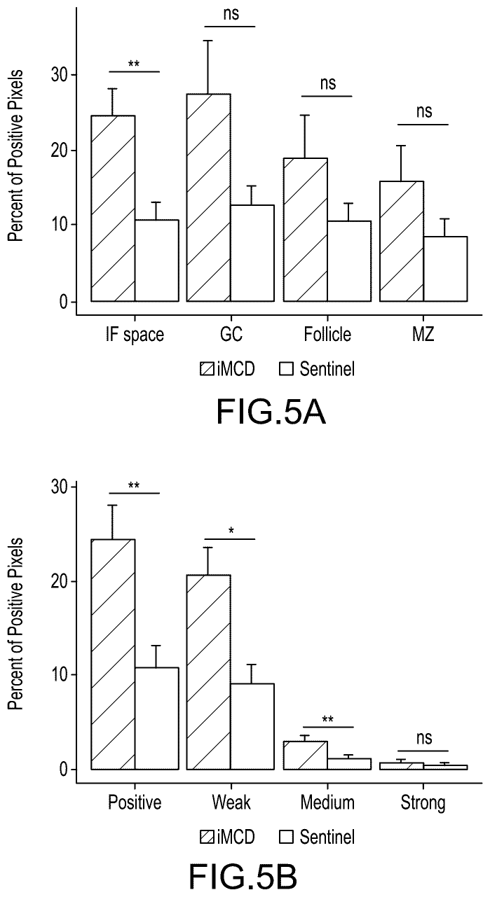

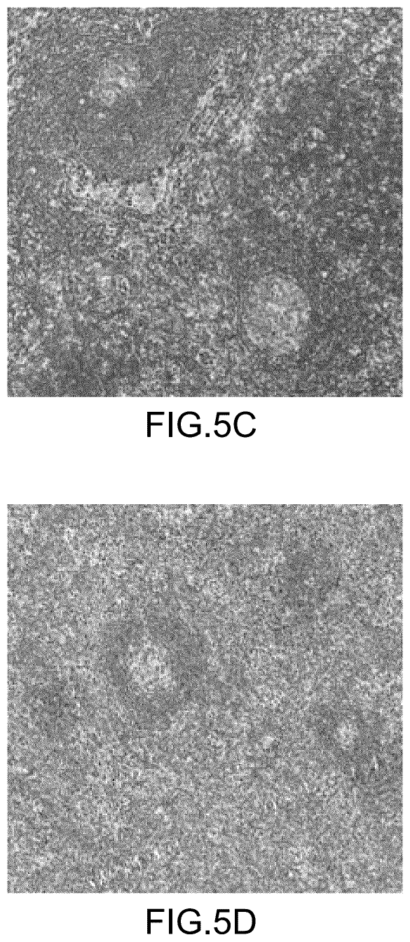

Given that IL-6 inhibition is not effective in siltuximab non-responders, IL-6-JAK-STAT3 signaling was not expected to be enriched in the serum proteome of siltuximab non-responders. To confirm activation of this pathway in the primary site of iMCD pathology, we performed IHC for phosphorylated-STAT3 (pSTAT3), an indicator of JAK-STAT3 activation, on iMCD lymph node tissue. We analyzed expression of pSTAT3 in 10 iMCD lymph nodes and 15 normal lymph nodes, as well as lymph nodes from 13 patients with HL as a positive control. As expected, pSTAT3 was significantly elevated in the interfollicular space of HL compared to normal controls (p=0,00022). We observed significantly increased pSTAT3 expression in the interfollicular space of iMCD lymph node tissue compared to normal (p=0.0037) and no significantly increased expression in the germinal centers (p=0.2610) (FIG. 5A). Weak and medium pSTAT3 intensity was significantly increased in the interfollicular space in iMCD compared to normal (weak p=0.014; medium p=0.0066; strong p=0.57) (FIG. 5B-D). Consistent with the enrichment analysis, these data suggest that pSTAT3 expression is increased in iMCD lymph node tissue and that JAK-STAT3 signaling is activated in iMCD tissue.

To investigate potential differences in the IL-6-JAK-STAT3 pathway between siltuximab responders and non-responders, we evaluated IL-6 and pSTAT3 IHC expression data from 51 and 48 patients, respectively, in the siltuximab treatment arm of the phase II study. Given the previous results, we hypothesized that pSTAT3 expression would be present at similar levels in non-responders and responders, suggesting that JAK-STAT3 pathway activation may be an iMCD driver in both responders and non-responders. Analysis of IL-6 and pSTAT3 expression did not reveal significant differences in expression between siltuximab responders and non-responders in any of the regions of the lymph node tissue that were quantified (IL-6 in germinal centers (p=0.56), mantle zone (p=0.96), and interfollicular space (p=0.34); pSTAT3 in germinal centers (p=0.86), mantle zone (p=0.98), and interfollicular space (p=1.0)). The lack of a difference in IL-6 or pSTAT3 expression between siltuximab responders and non-responders suggests that increased JAK-STAT3 pathway activation may occur in siltuximab non-responders secondary to another ligand independent of or in addition to IL-6 and may drive disease activity.

The present inventor has therefore identified and validated a novel subgroup of iMCD patients, herein called Cluster-1, with superior response to siltuximab and identified candidate therapeutic targets for siltuximab non-responders. Early identification of patients likely to respond to siltuximab and discovery of possible alternative treatments for non-responders are meaningful for patient care and represent unmet medical needs. These results represent the first validated predictive algorithm for response to siltuximab in iMCD. These seven proteins could form the basis for development of a clinical predictive signature. The association of these specific proteins with the Cluster-1 subgroup suggests important roles for plasma cells, antibodies, and dysregulated inflammation in patients who respond to siltuximab. IgE is a class of antibodies, IL-6 is a potent B cell differentiation and plasma cell growth factor, and iC3b is a complement component that can be induced through antibody complexes. Elevated SAP and Epo likely reflect reactive changes to increased systemic inflammation and inflammation-induced anemia, respectively.³³⁻³⁵ Both ApoE and AREG levels were negatively associated with the Cluster-1 patients. Interestingly, both are negative regulators of the immune system and inflammation.^{36,37} In fact, in autoimmune murine models, reduction of ApoE worsens autoimmune disease severity, and complete loss of ApoE induces rapid production of auto-antibodies, lymphoproliferation, and germinal center formation.³⁶

The validation of the proposed Cluster-1 subgroup is notable, particularly in iMCD, where samples are rare. Two different proteomic platforms and quantification techniques were utilized between the discovery and validation cohorts. Further, the validation cohort was comprised of patients from the phase I, dose-finding study of siltuximab,²¹ which included varying doses (35% of patients received a lower dose of siltuximab than was given in the phase II study), different inclusion and exclusion criteria from the phase II study, and didn't assess durable symptomatic response. Of note, 18 of 20 patients in the phase II study, who achieved lymph node response by Cheson criteria, also achieved durable symptomatic response, therefore, radiological lymph node response consistently identified responding patients in both studies. Despite these limitations and the relatively small sample size in the validation cohort, the association between Cluster-1 signature and siltuximab response in discovery (p=2.05×10⁻⁵) and validation (p=0.0757) cohorts suggest that this is a robust finding.

The proteomic data was further analyzed to identify candidate novel pathways and therapeutic targets. The enrichment analysis of proteomes from Cluster-1 responders identified IL-6-JAK-STAT3 signaling as a key pathway, demonstrating the potential for the platform and enrichment database to identify driver pathways. Surprisingly, IL-6-JAK-STAT3 signaling was also found to be significantly enriched among siltuximab non-responders in the enrichment analysis. Tissue-based IHC confirmed these results and revealed significantly increased pSTAT3 expression in the interfollicular space of iMCD lymph nodes compared to normal with no differences in IL-6 expression or pSTAT3 expression between responders and non-responders. The enrichment of IL-6-JAK-STAT3 signaling in iMCD serum proteomes, increased pSTAT3 expression in iMCD compared to normal, and lack of a difference in IL-6 and pSTAT3 expression between responders and non-responders suggest that the JAK-STAT3 pathway may still drive iMCD in siltuximab non-responders, either under the control of an activating ligand other than IL-6 or due to an aberration downstream of IL-6.

Based on the results of this study, targeting another aspect of the IL-6-JAK-STAT3 pathway with an agent such as ruxolitinib, a JAK1/2 inhibitor FDA-approved for myelofibrosis⁴⁰, may be potentially useful for siltuximab non-responders. JAK1/2 is a central node critical to STAT3 phosphorylation that is downstream of many potential driver cytokines. Ruxolitinib has demonstrated activity in other hyperinflammatory, cytokine-driven diseases, such as acute graft-versus-host disease⁴¹ and hemophagocytic lymphohistiocytosis⁴², by suppressing proinflammatory cytokines and reducing T cell proliferation through interrupting STAT signaling.

The enrichment analysis identified other candidate pathways that could contribute to the disease process in both siltuximab responders and non-responders. Many of these pathways can also be targeted with FDA-approved agents, such as TNFα, IFNγ, IL-2, and components of the allograft rejection signature. Only four of these agents have been reported in the iMCD literature.^{22-24,32} Drugs targeting IL-2-STATS signaling, enriched only among the siltuximab non-responders, and allograft rejection, enriched in both groups of iMCD patients, include cyclosporine, sirolimus, and tacrolimus, each of which has been reported to have potential activity in iMCD.^{23,24,32} TNFα signaling via NFκB, also enriched in both the siltuximab responders and non-responders, is another compelling target. TNFα is capable of inducing IL-6, VEGF, and JAK-STAT3 activation⁴³⁻⁴⁵ and could drive pSTAT3 through stimulating production of ligands other than IL-6.⁴⁶ In autoimmune diseases like RA, anti-TNFα decreases cytokine production, increases hemoglobin, and decreases inflammation.⁴⁷⁻⁴⁹ Considering these functions and proteomic overlap between iMCD and RA, anti-TNFα drugs should be further investigated as candidate drugs in siltuximab non-responders. Interestingly, the PI3K/Akt/mTOR pathway was not identified in GSEA analysis for this study. IL-6 signaling can activate both the JAK-STAT3 and PI3K/Akt/mTOR pathways.⁵⁰ In prior studies, the mTORC1 signaling pathway was found to be significantly enriched in iMCD, mTORC1 activation was significantly increased in iMCD lymph node tissue, and the mTOR inhibitor sirolimus has shown promise in the treatment of siltuximab non-responders.^{24,51} Further, interferon-β and IL-6 have been recently shown to induce increased mTOR activation in circulating immune cells from iMCD patients in remission compared to healthy controls, which could be abrogated with mTOR or JAK1/2 inhibition.⁵² It is possible that in some patients, there is an aberration downstream or independent of IL-6 that affects the PI3K/Akt/mTOR pathway and can be abrogated with mTOR inhibition and/or JAK1/2 inhibition.

### REFERENCES

Superscripted numbers in the preceding text of Example 1 refer to the correspondingly numbered references as follows:
1. Dispenzieri A, Armitage J O, Loe M J, et al. The clinical spectrum of Castleman's disease. Am. J. Hematol. 2012; 87(11):997-1002.
2. Melikyan A L, Egorova E K, Kovrigina A M, et al. [Clinical and morphological features of different types of Castleman's disease]. Ter. Arkh. 2015; 87(7):64-71.
3. Munshi N, Mehra M, van de Velde H, et al. Use of a claims database to characterize and estimate the incidence rate for Castleman disease. Leuk. Lymphoma. 2015; 56(5):1252-1260.
4. Fajgenbaum D C. Novel insights and therapeutic approaches in idiopathic multicentric Castleman disease. Blood. 2018; 132(22):2323-2330.
Fajgenbaum D C, Uldrick T S, Bagg A, et al. International, evidence-based consensus diagnostic criteria for HHV-8—negative/idiopathic multicentric Castleman disease. Blood. 2017; 129(12):1646-1658.
6. Yoshizaki K, Matsuda T, Nishimoto N, et al. Pathogenic significance of interleukin-6 (IL-6/BSF-2) in Castleman's disease. Blood. 1989; 74(4):1360-7.
7. Beck J T, Hsu S M, Wijdenes J, et al. Brief report: alleviation of systemic manifestations of Castleman's disease by monoclonal anti-interleukin-6 antibody. N Engl. J. Med. 1994; 330(9):602-5.
8. Heinrich P C, Behrmann I, Müller-Newen G, Schaper F, Graeve L. Interleukin-6-type cytokine signalling through the gp130/Jak/STAT pathway. Biochem. J. 1998; 334(2):297-314.
9. Nishimoto N, Kanakura Y, Aozasa K, et al. Humanized anti-interleukin-6 receptor antibody treatment of multicentric Castleman disease. Blood. 2005; 106(8):2627-2632.
10. van Rhee F, Wong R S, Munshi N, et al. Siltuximab for multicentric Castleman's disease: a randomised, double-blind, placebo-controlled trial. Lancet. Oncol. 2014; 15(9):966-74.
11. van Rhee F, Voorhees P, Dispenzieri A, et al. International, evidence-based consensus treatment guidelines for idiopathic multicentric Castleman disease. Blood. 2018; blood-2018-07-862334.
12. Casper C, Chaturvedi S, Munshi N, et al. Analysis of inflammatory and anemia-related biomarkers in a randomized, double-blind, placebo-controlled study of siltuximab (anti-IL6 monoclonal antibody) in patients with multicentric castleman disease. Clin. Cancer Res. 2015; 21(19):4294-42304.
13. Liu A Y, Nabel C S, Finkelman B S, et al. Idiopathic multicentric Castleman's disease: a systematic literature review. Lancet Haematol. 2016; 3(4):e163-75.
14. Fujimoto S, Koga T, Kawakami A, et al. Tentative diagnostic criteria and disease severity classification for Castleman disease: A report of the research group on Castleman disease in Japan. Mod. Rheumatol. 2018; 28(1):161-167.
Cheson B D, Pfistner B, Juweid M E, et al. Revised response criteria for malignant lymphoma. J. Clin. Oncol. 2007; 25(5):579-586.
16. Gold L, Ayers D, Bertino J, et al. Aptamer-based multiplexed proteomic technology for biomarker discovery. PLoS One. 2010; 5(12):.
17. Ganz P, Heidecker B, Hveem K, et al. Development and Validation of a Protein-Based Risk Score for Cardiovascular Outcomes Among Patients With Stable Coronary Heart Disease. JAMA. 2016; 315(23):2532-41.
18. Schwarz E, Izmailov R, Spain M, et al. Validation of a blood-based laboratory test to aid in the confirmation of a diagnosis of schizophrenia. 2010.
19. Subramanian A, Tamayo P, Mootha V K, et al. Gene set enrichment analysis: A knowledge-based approach for interpreting genome-wide expression profiles. Proc. Natl. Acad. Sci. U S. A. 2005; 102(43):15545-15550.
20. Döring C, Hansmann M L, Agostinelli C, et al. A novel immunohistochemical classifier to distinguish Hodgkin lymphoma from ALK anaplastic large cell lymphoma. Mod. Pathol. 2014; 27(10):1345-1354.
21. van Rhee F, Fayad L, Voorhees P, et al. Siltuximab, a novel anti-interleukin-6 monoclonal antibody, for Castleman's disease. J. Clin. Oncol. 2010; 28(23):3701-3708.
22. El-Osta H, Janku F, Kurzrock R. Successful treatment of Castleman's disease with interleukin-1 receptor antagonist (Anakinra). Mol. Cancer Ther. 2010; 9(6):1485-1488.
23. Takasawa N, Sekiguchi Y, Takahashi T, et al. A case of TAFRO syndrome, a variant of multicentric Castleman's disease, successfully treated with corticosteroid and cyclosporine A. Mod. Rheumatol. 2019; 29(1):198-202.
24. Fajgenbaum D C, Langan R-A, Sada Japp A, et al. Identifying and targeting pathogenic PI3K/AKT/mTOR signaling in IL-6-blockade-refractory idiopathic multicentric Castleman disease. J. Clin. Invest. 2019;
25. Yamaga Y, Tokuyama K, Kato T, et al. Successful Treatment with Cyclosporin A in Tocilizumab-resistant TAFRO Syndrome. Intern. Med. 2016; 55(2):185-90.
26. Inoue M, Ankou M, Hua J, et al. Complete resolution of TAFRO syndrome (thrombocytopenia, anasarca, fever, reticulin fibrosis and organomegaly) after immunosuppressive therapies using corticosteroids and cyclosporin A: a case report. J. Clin. Exp. Hematop. 2013; 53(1):95-9.
27. Konishi Y, Takahashi S, Nishi K, et al. Successful Treatment of TAFRO Syndrome, a Variant of Multicentric Castleman's Disease, with Cyclosporine A: Possible Pathogenetic Contribution of Interleukin-2. Tohoku J. Exp. Med. 2015; 236(4):289-95.
28. Noda Y, Saka Y, Kato A, Mimura T, Naruse T. Successful rituximab treatment of TAFRO syndrome with pathological findings of glomerular endothelial damage. Clin. Nephroi. Case Stud. 2018; 6:16-20.
29. Nishioka H, Takegawa H, Kamei H. Disseminated cryptococcosis in a patient taking tocilizumab for Castleman's disease. J. Infect. Chemother. 2018; 24(2):138-141.
30. Takayama Y, Kubota T, Ogino Y, et al. TAFRO syndrome with disseminated intravascular coagulation successfully treated with tocilizumab and recombinant thrombomodulin. Intern. Med. 2018; 57(9):1291-1296.
31. Nakamura Y, Iwamoto K, Fujisawa Y, et al. Multiple haemangiomas in TAFRO syndrome with atypical features of glomeruloid and epithelioid haemangiomas. Eur. J. Dermatology. 2018; 28(3):371-372.
32. Shirai T, Onishi A, Waki D, Saegusa J, Morinobu A. Successful treatment with tacrolimus in TAFRO syndrome: two case reports and literature review. Medicine (Baltimore). 2018; 97(23):e11045.
33. Pepys M B, Baltz M, Gomer K, Davies A J, Doenhoff M. Serum amyloid P-component is an acute-phase reactant in the mouse. Nature. 1979; 278(5701):259-61.
34. Strachan A F, Johnson P M. Protein SAP (serum amyloid P-component) in Waldenstrom's macroglobulinaemia, multiple myeloma and rheumatic diseases. J. Clin. Lab. Immunol. 1982; 8(3):153-6.
35. Birgegard G, Hallgren R, Caro J. Serum erythropoietin in rheumatoid arthritis and other inflammatory arthritides: relationship to anaemia and the effect of anti-inflammatory treatment. Br. J. Haematol. 1987; 65(4):479-483.
36. Centa M, Prokopec K E, Garimella M G, et al. Acute Loss of Apolipoprotein E Triggers an Autoimmune Response That Accelerates Atherosclerosis. Arterioscler. Thromb. Vasc. Biol. 2018; 38(8):e145-e158.
37. Zaiss D M W, Gause W C, Osborne L C, Artis D. Emerging functions of amphiregulin in orchestrating immunity, inflammation, and tissue repair. Immunity. 2015; 42(2):216-226.
38. Kurzrock R, Redman J, Cabanillas F, et al. Serum Interleukin 6 Levels Are Elevated in Lymphoma Patients and Correlate with Survival in Advanced Hodgkin's Disease and with B Symptomsl. 1993.
39. Kim S J, Kang H J, Dong-Yeop S, et al. The Efficacy of JAK2 Inhibitor in Heavily Pretreated Classical Hodgkin Lymphoma: A Prospective Pilot Study of Ruxolitinib in Relapsed or Refractory Classical Hodgkin Lymphoma and Primary Mediastinal Large B-Cell Lymphoma. Blood. 2016; 128(22):.
40. Verstovsek S, Mesa R A, Gotlib J, et al. A double-blind, placebo-controlled trial of ruxolitinib for myelofibrosis. N. Engl. J. Med. 2012; 366(9):799-807.
41. Spoerl S, Mathew N R, Bscheider M, et al. Activity of therapeutic JAK 1/2 blockade in graft-versus-host disease. Blood. 2014; 123(24):3832-3842.
42. Broglie L, Pommert L, Rao S, et al. Ruxolitinib for treatment of refractory hemophagocytic lymphohistiocytosis. Blood Adv. 2017; 1(19):1533-1536.
43. De Cesaris P, Starace D, Riccioli A, et al. Tumor necrosis factor-alpha induces interleukin-6 production and integrin ligand expression by distinct transduction pathways. J. Biol. Chem. 1998; 273(13):7566-71.
44. Cha H-S, Bae E-K, Koh J-H, et al. Tumor necrosis factor-alpha induces vascular endothelial growth factor-C expression in rheumatoid synoviocytes. J. Rheumatol. 2007; 34(1): 16-9.
Guo D, Dunbar J D, Yang C H, Pfeffer L M, Donner D B. Induction of Jak/STAT signaling by activation of the type 1 TNF receptor. J Immunol. 1998; 160(6):2742-50.
46. Elliott M J, Maini R N, Feldmann M, et al. Randomised double-blind comparison of chimeric monoclonal antibody to tumour necrosis factor alpha (cA2) versus placebo in rheumatoid arthritis. Lancet (London, England). 1994; 344(8930):1105-10.
47. Taylor P C, Peters A M, Paleolog E, et al. Reduction of chemokine levels and leukocyte traffic to joints by tumor necrosis factor alpha blockade in patients with rheumatoid arthritis. Arthritis Rheum. 2000; 43(1):38-47.
48. Davis D, Charles P J, Potter A, et al. Anaemia of chronic disease in rheumatoid arthritis: In vivo effects of tumour necrosis factor α blockade. Br. J. Rheumatol. 1997; 36(9):950-956.
49. Paleolog E M, Young S, Stark A C, et al. Modulation of angiogenic vascular endothelial growth factor by tumor necrosis factor alpha and interleukin-1 in rheumatoid arthritis. Arthritis Rheum. 1998; 41(7):1258-65.
50. Zegeye M M, Lindkvist M, Fälker K, et al. Activation of the JAK/STAT3 and PI3K/AKT pathways are crucial for IL-6 trans-signaling-mediated proinflammatory response in human vascular endothelial cells. Cell Commun. Signal. 2018; 16(1):.
51. Arenas D J, Floess K, Kobrin D, et al. Increased mTOR activation in idiopathic multicentric Castleman disease. Blood. 2020;
52. Pai R A L, Japp A S, Gonzalez M, et al. Type I IFN response associated with mTOR activation in the TAFRO subtype of idiopathic multicentric Castleman disease. JCI insight. 2020; 5(9):.

### Example 2—CXCL13 as Early Indicator of Response to iMCD Treatment

CXCL13, a key regulator of lymph node germinal center development, was recently found to be the most elevated cytokine in iMCD flare, but the clinical significance of this finding is not yet clear. Interleukin-6 (IL-6) is the known driver of pathogenesis in a portion of patients and the target of the only FDA-approved treatment, siltuximab. In the Phase II study of siltuximab (NCT01024036), one-third of patients met response criteria, which were assessed after a minimum of 48 weeks. Early indicators of response to siltuximab are urgently needed to inform clinicians about the likelihood of patient response to therapy, adjust treatments if needed, and identify novel therapeutic targets for siltuximab non-responders.

Methods. Clinical data and serum samples were collected as part of NCT01024036. We measured serum protein analytes in the 52 subjects who were treated with anti-IL6 therapy, as well as the 26 patients in the control arm, at day 1, day 8, and day 64 of therapy (infusions administered every 21 days). Serum samples from 44 healthy donors were also analyzed. Of the 1,305 analytes measured using SomaLogic SOMAscan, 1,178 passed QC and were included in analyses. Each analyte was log 2 transformed and capped at the 2.5th and 97.5^{th} percentiles. Response to anti-IL6 therapy was determined by independent review in NCT01024036. Data processing was performed using the Medidata Rave Omics machine learning platform and R v3.4.4.

Linear mixed effects models were used to detect whether kinetic changes in protein expression were associated with anti-IL6 response. Upon running the full model with the selected covariates, the p-values of the interaction between time point and response were used to test for differences between responders and nonresponders.

A separate model was fitted using each protein, and False Discovery Rates (FDR) were estimated by the Benjamini-Hochberg method with alpha <0.05.

Results. Seven days after siltuximab was first administered (day 8), 9 proteins were significantly different between responders and non-responders: IgA, BCMA, NPS-PLA2, ART, IL-18 BPa, CD5L, b2-Microglobulin, CXCL13, and NRP1. All 9 of these proteins were significantly decreased in responders compared to non-responders. At day 64, the number of significantly different proteins increased to 121, including 8 of the 9 proteins from day 8; NPS-PLA2 did not achieve significance at day 64. This result indicates that there may be early indicators of response in serum as early as day 8.

Given that CXCL13 was recently discovered as a key cytokine in iMCD, the early and significant decline of CXCL13 in responders versus non-responders was highly notable (Day 8: FDR=0.02, Day 64: FDR=0.005).

Prior to treatment, CXCL13 was significantly higher in this cohort of iMCD patients than in a group of age-matched healthy donors (p=8.19e-09). By day 64, CXCL13 levels in siltuximab responders decreased to levels approaching the healthy donor range but remained elevated in non-responders and placebo patients.

TABLE 2
Demographic characteristics for 52 patients treated
with siltuximab and 26 placebo arm patients

| | | Siltuximab-treated | | |
|---|---|---|---|---|
| | | Responder | Non-responder | Placebo |
| N | | 18 | 34 | 26 |
| Sex, N (%) | Female | 8 (44.4) | 14 (41.2) | 4 (15.4) |
| | Male | 10 (55.6) | 20 (58.8) | 22 (84.6) |
| Age | Mean (SD) | 52.2 (15.0) | 52.1 (12.7) | 53.8 (13.8) |
| | Missing | 0 | 5 | 2 |
| Baseline | Mean (SD) | 3.7 (2.5) | 1.5 (2.4) | 1.3 (1.6) |
| CHAP | | | | |
| Score | Missing | 1 | 1 | 2 |

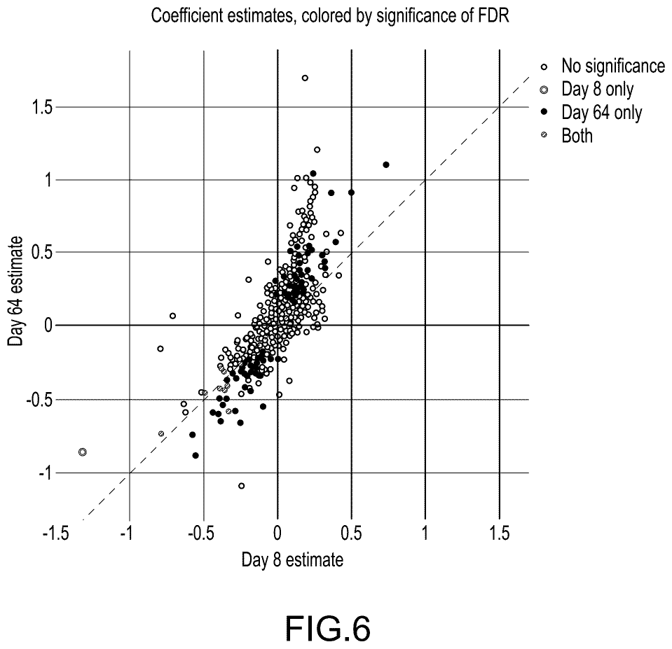

At day 8, proteins were significantly different in responders compared to non-responders (Table 3). At day 64, the number of significantly different proteins increased to 121, including 8 of the 9 proteins from day 8 (FIG. 6).

TABLE 3
Significant Proteins

| Nine Proteins | Ten Most Significant | Largest Fold |
|---|---|---|
| Significant at Day 8 | Proteins at Day 64 | Change at Day 64 |
| IgA* | IgA* | Myokinase, human |
| NPS-PLA2 | CD36 ANTIGEN | IL-6 |
| CD5L* | IL-6 | PPAC |
| CXCL13* | Growth hormone receptor | UBE2N |
| B2-Microglobulin* | FCG2B | Aflatoxin B1 aldehyde |
| | | reductase |
| ART* | CRDL1 | 41 |
| IL-18 BPa* | IGFBP-2 | WNK3 |
| NRP1* | C7 | I-TAC |
| BCMA* | NovH | Midkine |
| — | IL-4 | CXCL13* |

*Significant at both time points
** Italicized text designates positive effect, non-italicized indicates negative effect

Conclusions. This analysis represents the first use of high-quality serum proteomics data to study early indicators of response to treatment in a rare hyperinflammatory, lymphoproliferative disorder. The decline in CXCL13 levels in responders and continued elevation in non-responders suggests that CXCL13 is downstream of IL-6 in responders and independent of IL-6 signaling in non-responders. CXCL13, along with several other proteins that demonstrated significant decline by day 8 including IgA and beta2-microglobulin, can be routinely measured and could serve as a panel that indicates the likelihood of response soon after commencing therapy, if validated in a separate cohort. These proteins also provide a more continuous scale of response than traditional outcome measures. Given that iMCD may have a sudden and severe onset, early indicators of response to anti-IL6 therapy are critical for timely treatment administration.

### Example 3—CXCL13 as Diagnostic Biomarker for iMCD

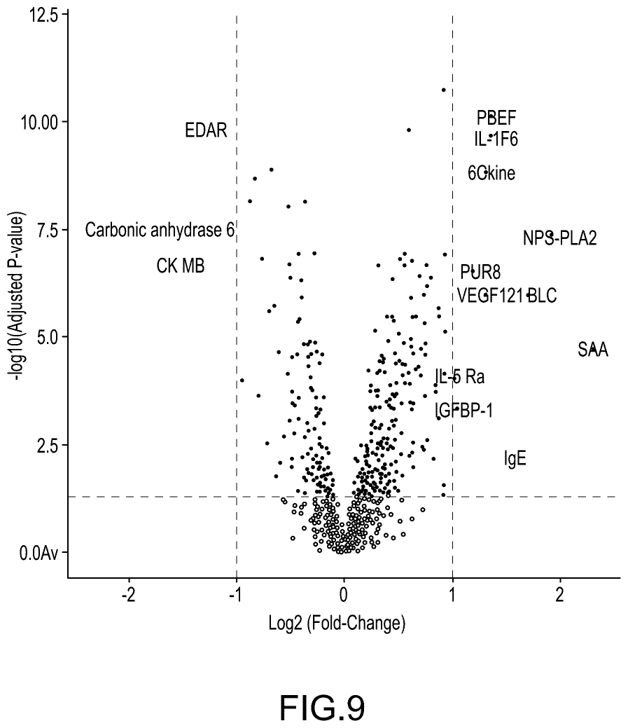

Among 1,178 proteins measured in 88 iMCD patients and 44 healthy controls, CXCL13 is the third most up-regulated protein (along with 2 non-specific markers of inflammation) and the top cytokine in the blood of iMCD patients compared to healthy controls (FIG. 9).

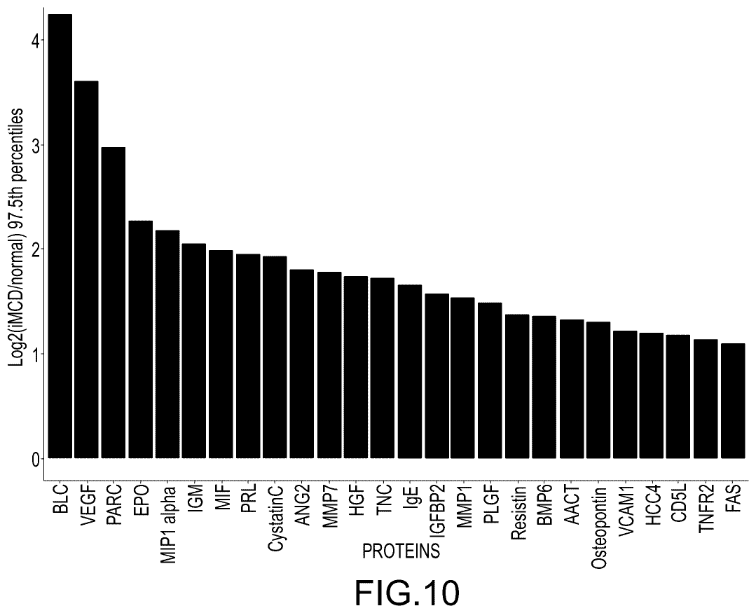

In a validation cohort of 23 iMCD patients where 190 analytes were measured, the present inventor found that CXCL13 was the top up-regulated protein when comparing the 97.5^{th} percentile of iMCD patients versus the top 97.5^{th} percentile of healthy controls (FIG. 10).

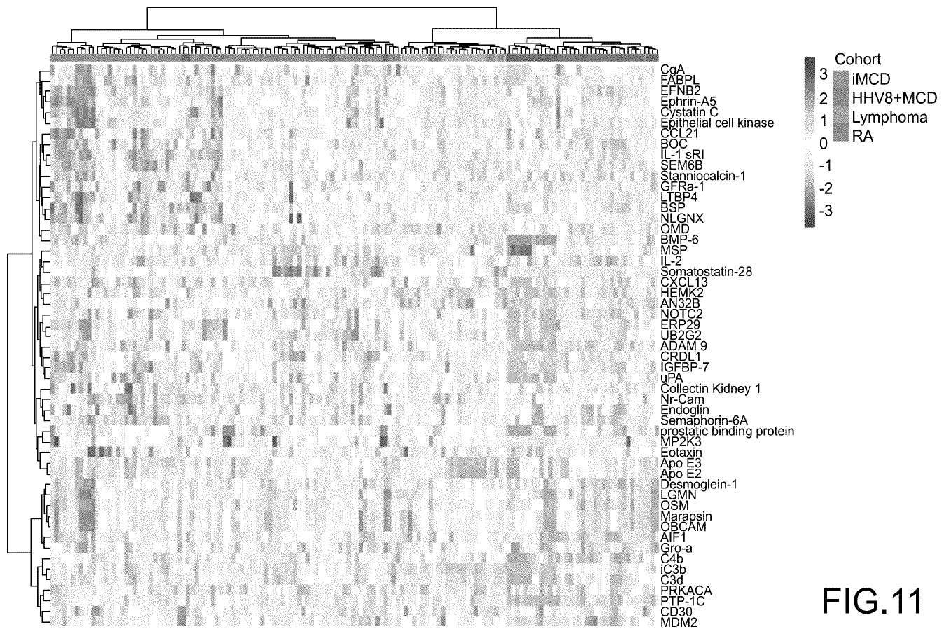

Differential expression analysis of 1,178 proteins between iMCD (N=88) and diseases with overlapping clinico-pathology, including rheumatoid arthritis (N=20), HHV-8-associated MCD (N=20), and Hodgkin lymphoma (N=20) revealed that CXCL13 was one of two proteins that differed significantly between each of the disease groups and was increased by two-fold in iMCD versus healthy controls (FIG. 11).

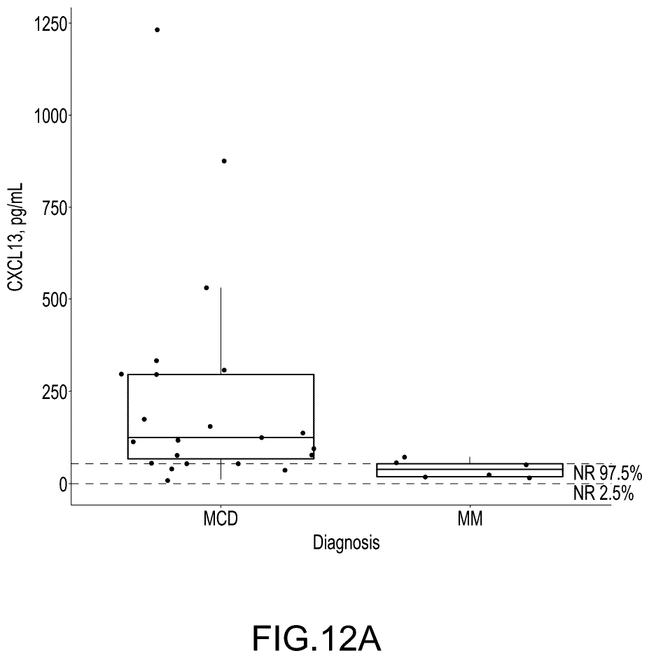

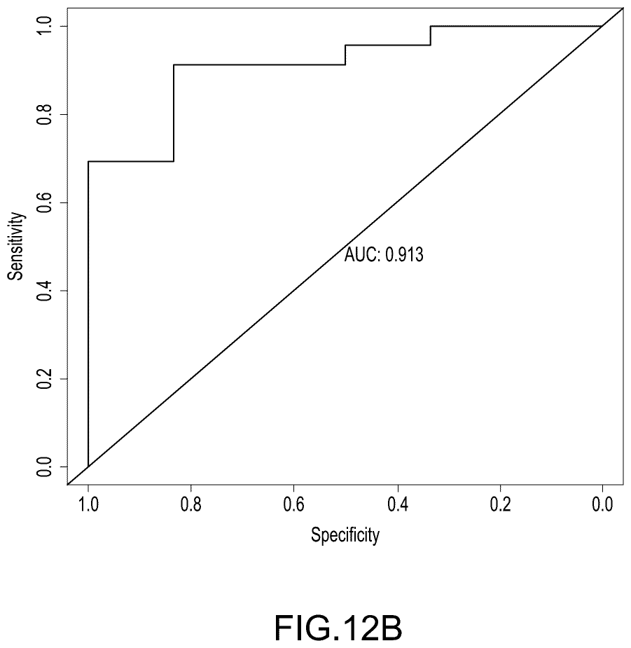

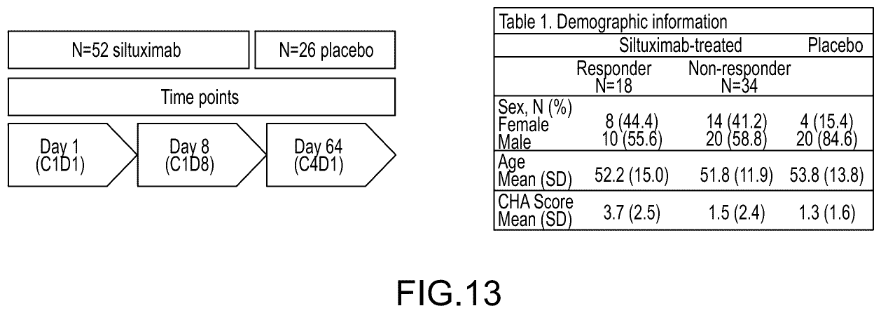

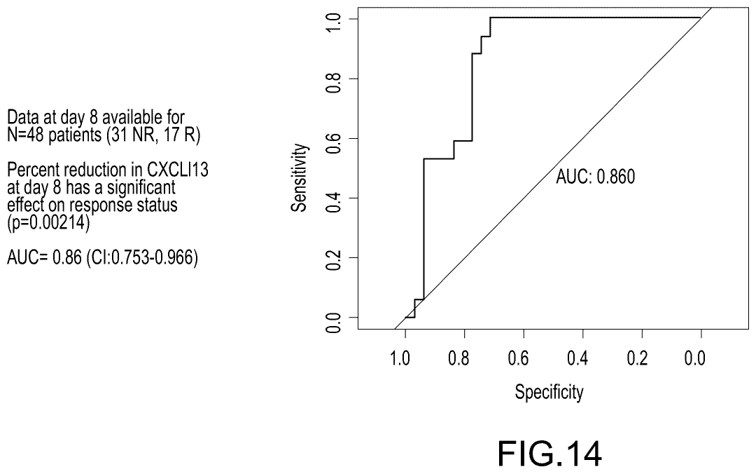

In the validation cohort of 23 iMCD patients, it was found that levels of CXCL13 in iMCD were nearly four-fold greater than that of a small cohort of multiple myeloma (N=6) patients and that having iMCD was significantly associated with higher CXCL13 (p=0.007) (FIG. 12).

Overall, these proteomics data, which include proteomic quantification on orthogonal platforms from both relative and absolute quantification and a combined total of over 100 iMCD patients, 44 healthy donors, and 66 related disease patients in two independent cohorts, have demonstrated strong and consistent upregulation of serum CXCL13 in iMCD, indicating that it can represent a clinically-relevant diagnostic biomarker relative to normal and various disease states. This is extremely important for patients and clinicians to have a blood-based test to support the diagnosis of iMCD (of note, no clinical CXCL13 assays are currently available).

### Example 4—Additional Information Regarding Evaluation of CXCL13 as Early Indicator of Response to Siltuximab

Given that interrogation of the iMCD proteome helped identify CXCL13 as a potential diagnostic biomarker, it was next sought to interrogate these data to explore another major unmet need: early indicators of response to siltuximab. If there were significant differences in proteomic levels shortly after siltuximab between responders versus non-responders, clinical testing of these proteins could be performed to determine if patients are likely to respond to siltuximab.

For this analysis, the present inventor looked across the 1,178 proteins measured in 79 iMCD patients in the phase II siltuximab trial collected at day 1 of treatment (pretreatment), day 8 (cycle 1 day 8), and day 64 (cycle 4 day 1).

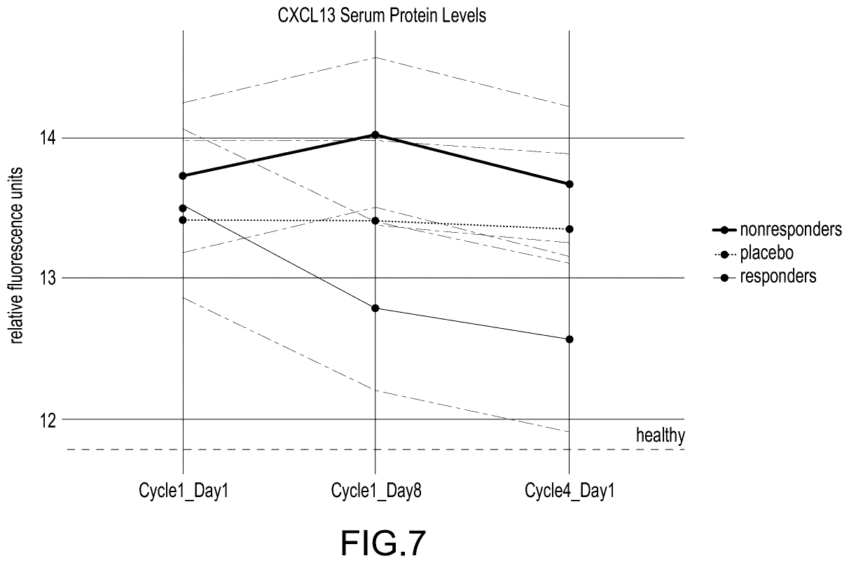

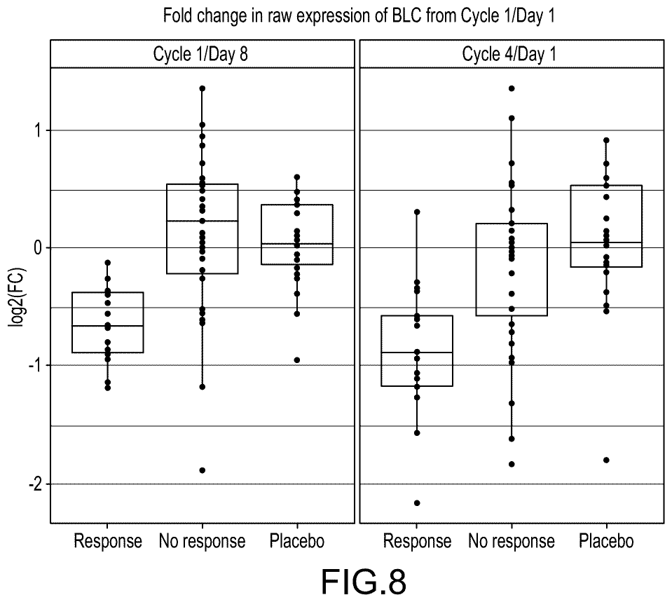

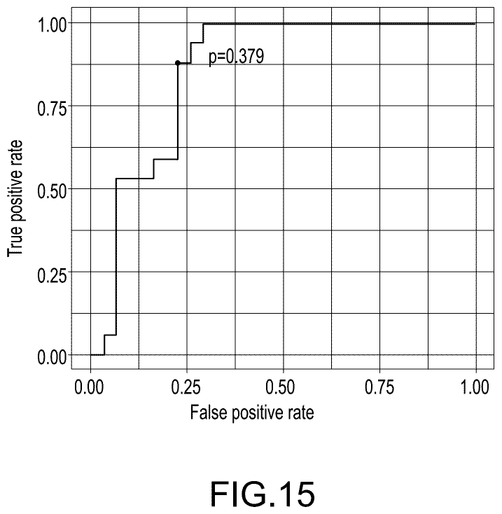

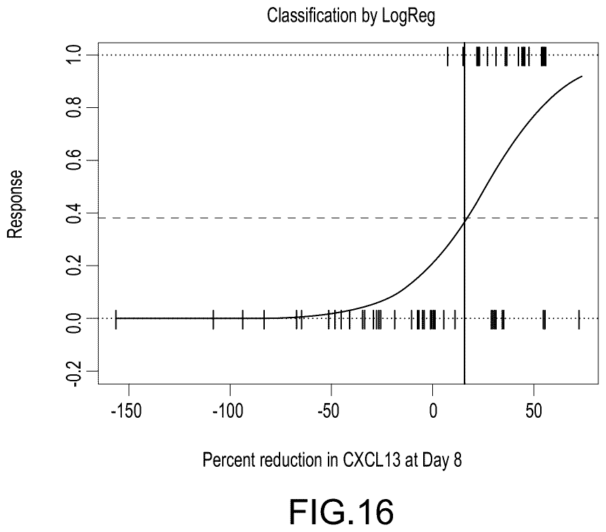

CXCL13 was one of 9 proteins significantly different between responders and non-responders (FDR <0.05) at day 8 (FIGS. 6-8 & 13). A reporter operator curve was generated to identify 17% as the optimal reduction in CXCL13 levels to maximize sensitivity and specificity of this indicator (FIGS. 7 & 14). Specifically, if a patient has a 17% or greater reduction in CXCL13 levels, then the patient is highly likely to respond (FIGS. 15-16, Table 4). The proteins from patients in the discovery cohort described supra were quantified using a relative quantification while the protein quantification in the validation described in Table 4 utilized absolute quantification.

TABLE 4
Validation Cohort - CXCL13 Levels

| | Responders | Non-Responders |
|---|---|---|
| | (N = 10) | (N = 13) |
| CXCL13, pg/ml | | |
| Baseline, Median (IQR) | 116 (57, 258) | 155 (79, 293) |
| Day 22/29, Median (IQR) | 41.1 (23.5, 62) | 249 (73.1, 466) |
| Day 43, Median (IQR) | 94.4 (29.6, 147) | 218 (62, 371) |
| CXCL13, pg/ml | | |
| Baseline, Mean (SD) | 241 (361) | 234 (240) |
| Day 22/29, Mean (SD) | 71.5 (94.5) | 337 (381) |
| Day 43, Mean (SD) | 94.8 (70.6) | 229.1 (190.3) |

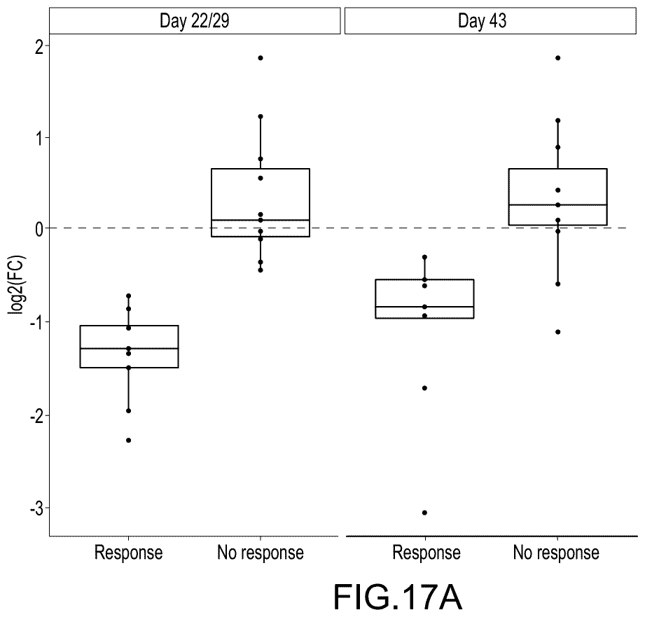

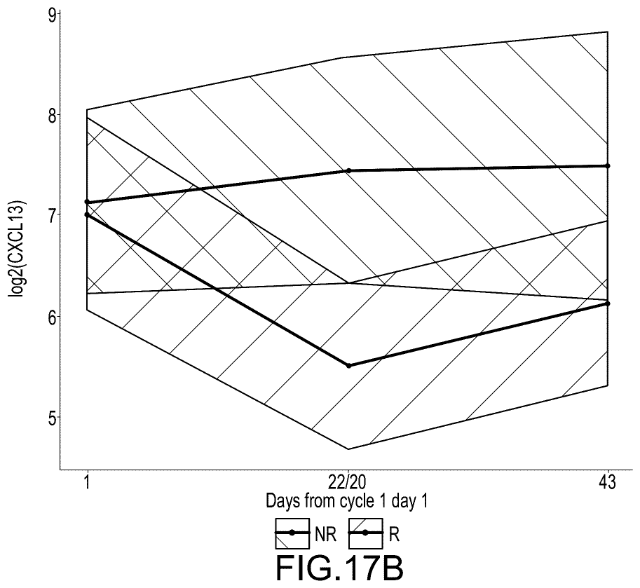

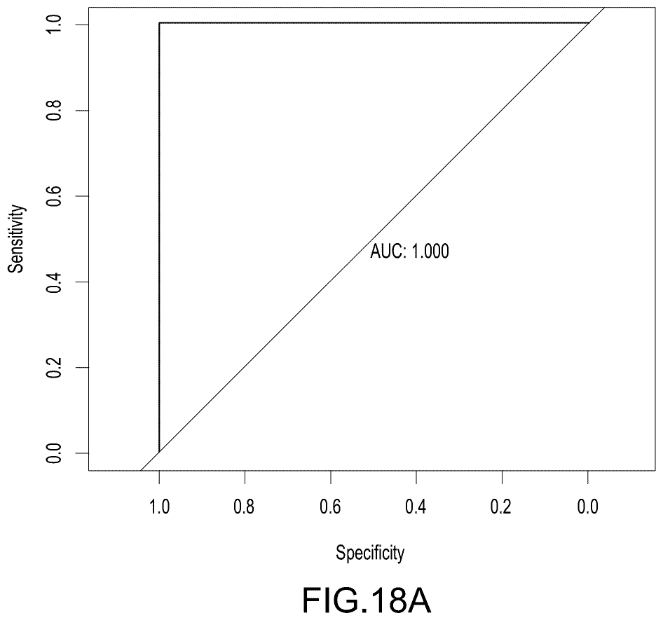

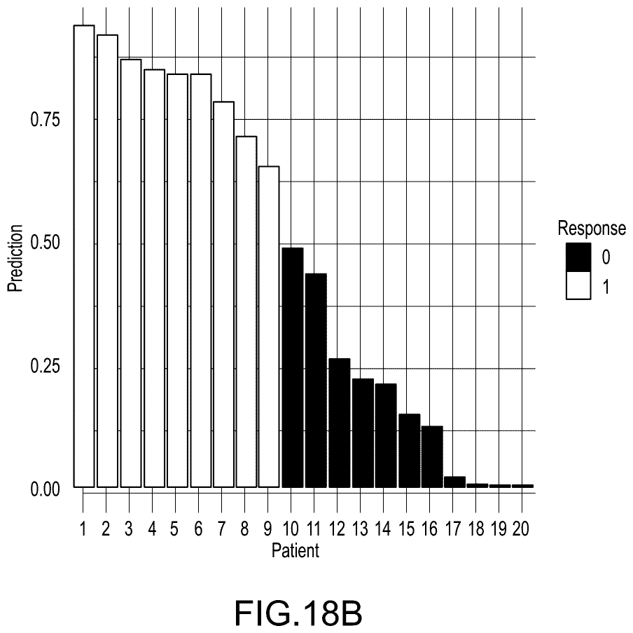

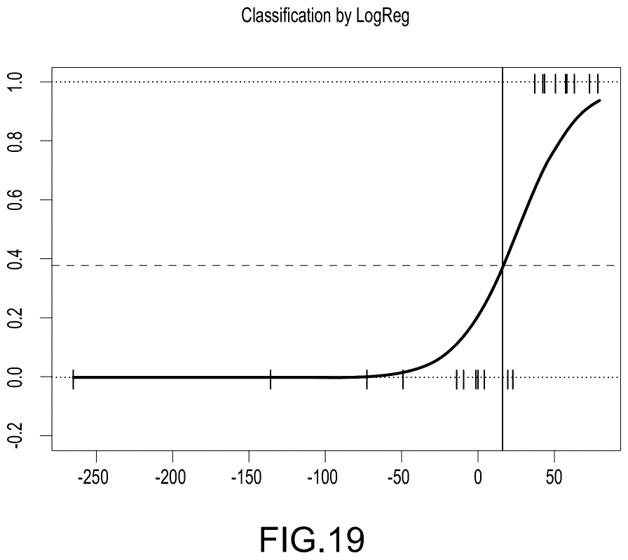

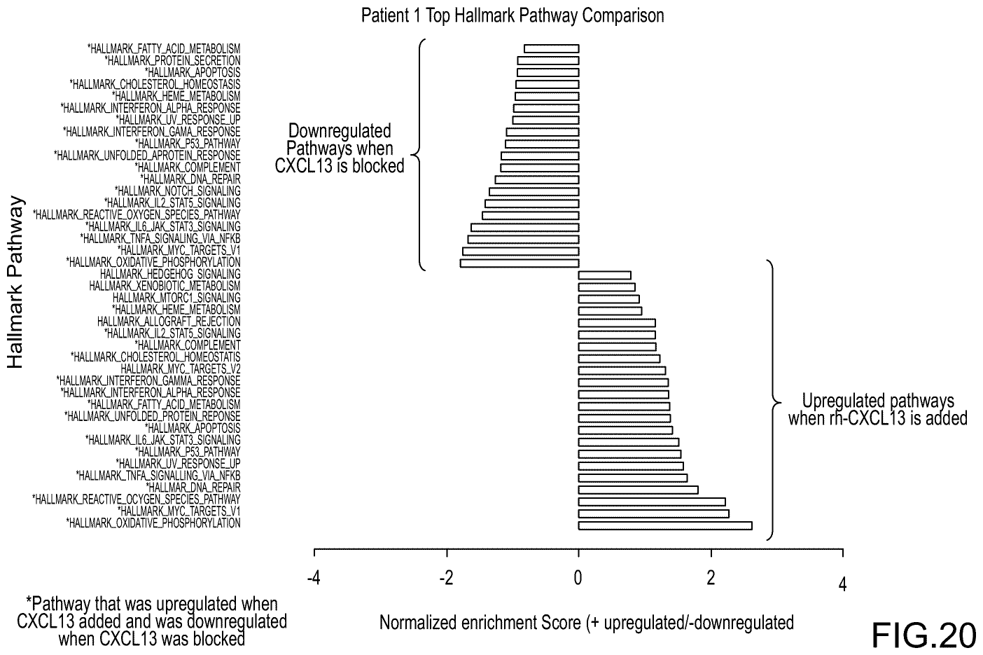

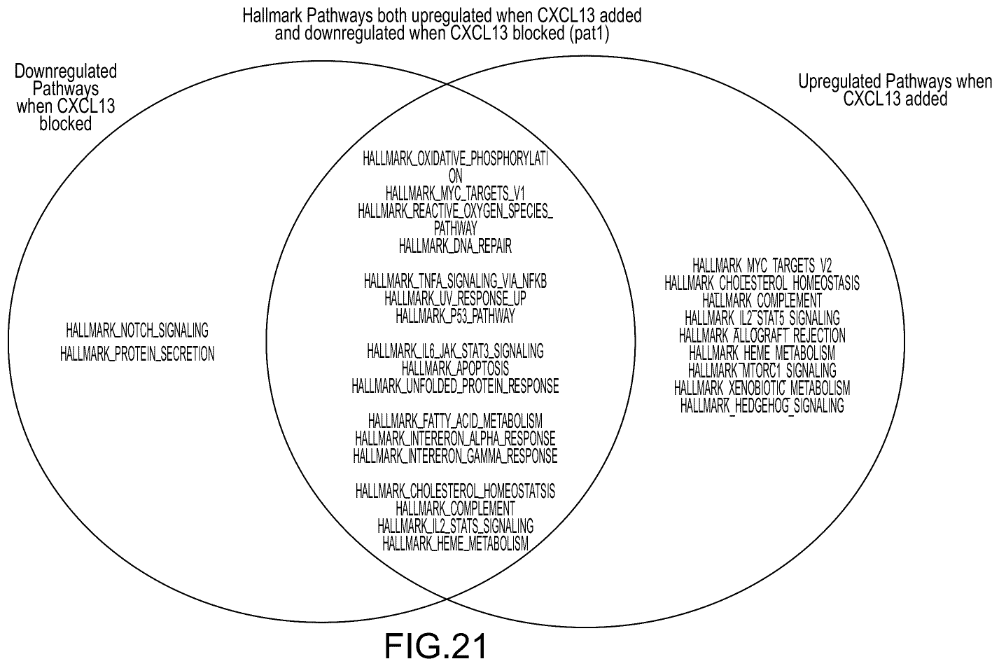

In the validation cohort of 23 siltuximab-treated iMCD patients whose samples were collected at day 1 (pretreatment), day 22 or 29 (day 22/29), and day 43, CXCL13 was the only protein significantly different at the first timepoint after siltuximab was started (day 22/29) (FIG. 17). Significantly, using reduction in CXCL13 levels completely separates responders from non-responders and using 17% as a threshold, identifies all patients who respond (FIGS. 18-19).

Together, these data indicate that CXCL13 is an early indicator of response to siltuximab which would be highly valuable to patients and physicians to help with determining if siltuximab treatment should be continued or not.

### Example 4—CXCL13 as a Therapeutic Target that can be Inhibited with Anti-CXCL13 Agents and/or Anti-CXCR5 Agents

The aforementioned correlative data strongly suggest that CXCL13 is critical to pathogenesis and a therapeutic target that could alleviate disease activity if inhibited as it is very elevated, different from related diseases, and declines rapidly in patients benefiting from anti-IL-6 therapy but remains elevated in non-responders.

It was hypothesized by the present inventor that CXCL13 causes phenotypic changes in CXCR5+ cells that lead to additional cytokine production and chemotaxis to improper locations of the lymph node and vital organs, leading to organ dysfunction and death.

An in vitro experiment was performed that included flow sorting CD19+CD27-IgD+B cells from peripheral blood mononuclear cells (PBMCs) derived from an iMCD patient and treating the CD19+CD27-IgD+B cells with either (A) serum only, (B) serum plus recombinant human CXCL13 (rhCXCL13) to assess the effects of CXCL13 on iMCD patient B cells, or (C) serum, rhCXCL13, and anti-CXCL13 (anti-CXCL13) antibody to assess the effects of this blocking antibody. Each of these samples had geneset enrichment analysis performed of the Hallmark pathways on RNA sequencing data.

Multiple pathways previously found to be up-regulated in iMCD patient samples (IL6-JAK-STAT3, mTORC1, Interferon alpha response, interferon gamma response, complement, TNFα signaling via NFKb) were found to be up-regulated in the samples treated with serum and rhCXCL13 compared to serum only, indicating that CXCL13 can induce these pathways critical to iMCD pathogenesis. These pathways were also down-regulated in the samples treated with rhCXCL13 and anti-CXCL13 compared to serum only, indicating that anti-CXCL13 can effectively abrogate these pathogenic

What is claimed:

1. A method for assigning a subject having idiopathic multicentric Castleman disease (iMCD) to a group having a higher or lower probability of responding to treatment for iMCD comprising:
comparing the amount of CXCL13 in a biological fluid obtained from the subject following commencement of the treatment to the amount of CXCL13 in a biological fluid obtained from the subject prior to commencement of the treatment; and,
assigning the subject to a group having a higher probability of responding to the treatment if the amount of CXCL13 in the biological fluid obtained from the subject following commencement of the treatment represents a significant downward deviation relative to the amount of CXCL13 in the biological fluid obtained from the subject prior to commencement of the treatment.

2. The method according to claim 1, wherein the biological fluid obtained from the subject following commencement of the treatment was obtained about one week following commencement of the treatment.

3. The method according to claim 1 or claim 2, wherein the treatment comprises anti-IL-6 therapy.

4. The method according to any preceding claim, wherein the treatment comprises administration of siltuximab to the subject.

5. The method according to any preceding claim, wherein when the subject is assigned to a group having a lower probability of responding to the treatment, reducing or ceasing the treatment following the assignment.

6. The method according to any preceding claim, wherein when the subject is assigned to a group having a higher probability of responding to the treatment, continuing the treatment following the assignment.

7. The method according to any preceding claim, wherein when the amount of CXCL13 in the biological fluid obtained from the subject following commencement of the treatment represents a significant downward deviation relative to the amount of CXCL13 in the biological fluid obtained from the subject prior to commencement of the treatment and the significant downward deviation is about 17% or greater, assigning the subject to a group having a higher probability of responding to the treatment.

8. The method according to any preceding claim, wherein when the amount of CXCL13 in the biological fluid obtained from the subject following commencement of the treatment represents a significant downward deviation relative to the amount of CXCL13 in the biological fluid obtained from the subject prior to commencement of the treatment and the significant downward deviation is about 17% or greater, assigning the subject to a group having a higher probability of responding to the treatment.

9. A method of treating idiopathic multicentric Castleman disease (iMCD) in a subject in need thereof comprising administering to the subject an inhibitor of CXCL13.

10. The method according to claim 9, further comprising administering to the subject a further treatment for iMCD at least partially during administration of the CXCL13 to the subject, at least partially prior to administration of the inhibitor of CXCL13 to the subject, at least partially following administration of the inhibitor of CXCL13 to the subject, or any combination thereof.

11. The method according to any one of claims 9-10, further comprising administering to the subject an inhibitor of IL-6 at least partially during administration of the CXCL13 to the subject, at least partially prior to administration of the inhibitor of CXCL13 to the subject, at least partially following administration of the inhibitor of CXCL13 to the subject, or any combination thereof.

12. The method according to any one of claims 9-11, further comprising administering to the subject an inhibitor of CXCR5 at least partially during administration of the CXCL13 to the subject, at least partially prior to administration of the inhibitor of CXCL13 to the subject, at least partially following administration of the inhibitor of CXCL13 to the subject, or any combination thereof.

13. The method according to claim 12, wherein the inhibitor of CXCR5 is SAR113244 antibody.

14. The method according to any one of claims 9-13, further comprising administering to the subject an inhibitor of JAK protein or of the JAK/STAT3 pathway at least partially during administration of the CXCL13 to the subject, at least partially prior to administration of the inhibitor of CXCL13 to the subject, at least partially following administration of the inhibitor of CXCL13 to the subject, or any combination thereof.

15. The method according to claim 14, wherein the further treatment for iMCD is sirolimus.

16. A method of treating idiopathic multicentric Castleman disease (iMCD) in a subject in need thereof comprising administering to the subject an inhibitor of CXCR5.

17. The method according to claim 16, wherein the inhibitor of CXCR5 is SAR113244 antibody.

18. The method according to claim 16 or claim 17, further comprising administering to the subject a further treatment for iMCD during administration of the inhibitor of CXCR5 to the subject.

19. The method according to claim 18, wherein the further treatment for iMCD is an inhibitor of IL-6.

20. The method according to claim 18, wherein the further treatment for iMCD is an inhibitor of JAK protein or of the JAK/STAT3 pathway.

21. A method for assigning a subject having idiopathic multicentric Castleman disease (iMCD) to a group having a higher or lower probability of responding to treatment for iMCD comprising:
comparing the amount of biomarkers comprising one or more of APO E, SAP, iC3b, AREG, IgE, IL-6, and Epo in a biological fluid obtained from the subject prior to commencement of the treatment to reference values of the one or more biomarkers; and,
assigning the subject to a group having a higher probability of responding to the treatment if the respective amounts of the one or more biomarkers in the biological fluid obtained from the subject prior to commencement of the treatment represent a significant upward deviation relative to the reference values for the one or more biomarkers.

22. A method for assigning a subject having idiopathic multicentric Castleman disease (iMCD) to a group having a higher or lower probability of responding to treatment for iMCD comprising:
measuring the amount of biomarkers comprising one or more of APO E, SAP, iC3b, AREG, IgE, IL-6, and Epo in a biological fluid obtained from the subject prior to commencement of the treatment; and,
assigning the subject to a group having a higher or lower probability of responding to treatment for iMCD using an optimized output of a function of the measured biomarkers in the biological fluid.

23. The method according to claim 22, wherein the function comprises respective optimized weighting coefficients for the measured amounts of the one or more biomarkers.

24. A method of treating idiopathic multicentric Castleman disease (iMCD) in a subject in need thereof comprising administering to the subject an inhibitor of the JAK-STAT3 pathway.

25. The method according to claim 24, comprising administering to the subject an inhibitor of JAK protein.

26. A method for assessing the absence or presence of iMCD in a subject comprising:
measuring an amount of CXCL13 in a biological fluid obtained from the subject,
comparing the measured amount of CXCL13 in the biological fluid to a reference value corresponding to an amount of CXCL13 signifying a lower or higher likelihood of a positive diagnosis of iMCD, and
assigning to the subject a higher likelihood of a positive diagnosis of iMCD if the measured amount of CXCL13 represents a significant upward deviation relative to the reference value of CXCL13 and a lower likelihood of a positive diagnosis of iMCD if the measured amount of CXCL13 does not represent a significant upward deviation relative to the reference value of CXCL13.

27. The method according to claim 26, wherein if the measured amount of CXCL13 from the biological fluid of the subject represents a significant upward deviation relative to the reference value of CXCL13, further comprising treating the subject for iMCD.
